(19) United States
(12) Patent Application Publication
(10) Pub. No.: US 2026/0196306 A1
(43) Pub. Date: Jul. 9, 2026

(54) SPATIAL BIOLOGY INFORMATICS INTEGRATION PORTAL WITH PROGRAMMABLE MACHINE LEARNING PIPELINE ORCHESTRATOR

(71) Applicant: Bruker Spatial Biology, Inc., Seattle, WA (US)

(72) Inventors: John BARTON, Seattle, WA (US); Seth BIBLER, Seattle, WA (US); Richard BOYKIN, Seattle, WA (US); Alexander BUELL, Seattle, WA (US); David HENDERSON, Seattle, WA (US); Michael MCKEAN, Seattle, WA (US); SanghaMithra KORUKONDA, Seattle, WA (US); Aster WARDHANI, Seattle, WA (US); April D. MUNN, Seattle, WA (US)

(73) Assignee: Bruker Spatial Biology, Inc., Seattle, WA (US)

(21) Appl. No.: 18/870,623

(22) PCT Filed: Jun. 2, 2023
(86) PCT No.: PCT/US2023/067821

§ 371 Date: Nov. 29, 2024

Related U.S. Application Data
(60) Provisional application No. 63/348,936, filed on Jun. 3, 2022.
(60) Provisional application No. 63/381,528, filed on Oct. 28, 2022.

(51) Int. Cl. G16B 45/00; G06F 3/0486; G16B 40/00; G16B 50/30

(52) U.S. Cl. CPC G16B 45/00; G06F 3/0486; G16B 50/30; G16B 40/00

(57) ABSTRACT

Described herein are platforms, systems, media, and methods for spatial biology informatics analysis utilizing an instrument interface communicatively coupled to one or more laboratory instruments; a software element configured to receive data, directly or indirectly, from the one or more laboratory instruments, wherein the data comprises biological image data and one or more of: genomic, proteomic, metabolomic, metagenomic, phenomic, and transcriptomic data; a software element configured to provide a pipeline orchestrator tool allowing a user to create, edit, manage, and execute analysis pipelines, wherein the pipeline orchestrator tool allows the user to link together modules and run them subsequently or in parallel to transform the received data; and a software element configured to generate a visualization of the data.

### CROSS REFERENCE

This application claims the benefit of PCT Application No. PCT/US2023/067821, filed Jun. 2, 2023, which claims the benefit of U.S. Provisional Patent Application No. 63/348,936, filed Jun. 3, 2022, and U.S. Provisional Patent Application No. 63/381,528, filed Oct. 28, 2022, each of which is entirely incorporated herein by reference for all purposes.

### BACKGROUND

Spatial biology is the study of tissues within their own 2D or 3D context. The field of spatial biology investigates the spatial location and organization of gene expression in situ within each cell and structure of a given tissue sample. Maintaining the spatial context of biological data is important for understanding how cells organize and interact with their surrounding environment to drive various biological functions.

### SUMMARY

Spatial transcriptomics and spatial proteomics can be used to study both healthy and diseased tissues by understanding the mechanisms behind the development, tissue heterogeneity, and therapeutic response. Historically, spatial patterns of gene expression were captured by immunohistochemistry (IHC) and in situ hybridization (ISH). These techniques provided researchers with the ability to visualize biological processes spatially by directly mapping the location of DNA, RNA, and protein molecules within tissues. However, these methods limited analysis to at most a handful of genes or proteins at a time.

Several new technologies have been developed that aim to combine gene expression data with spatial context. Most of the spatial transcriptomics technologies available today have been built on in situ and scRNA-seq and can be primarily categorized into (1) NGS-based approaches that encode positional information onto transcripts before sequencing and (2) imaging approaches based either on in situ sequencing (ISS) where transcripts are amplified and sequenced in the tissue or fluorescence in situ hybridization (FISH) where imaging probes are sequentially hybridized in the tissue. While both NGS- and imaging-based technologies are powerful, each has significant limitations. No single method can currently address all the desired parameters such as high throughput, sensitivity, resolution, and sample compatibility.

In one aspect, disclosed herein are systems comprising at least one processor and instructions executable by the at least one processor to provide a spatial biology informatics application comprising: an instrument interface communicatively coupled to one or more laboratory instruments; a software element configured to receive data, directly or indirectly, from the one or more laboratory instruments, wherein the data comprises biological image data and one or more of: genomic, proteomic, metabolomic, metagenomic, phenomic, and transcriptomic data; a software element configured to provide a pipeline orchestrator tool allowing a user to create, edit, manage, and execute analysis pipelines, wherein the pipeline orchestrator tool allows the user to link together modules and run them subsequently or in parallel to transform the received data; and a software element configured to generate a visualization of the received and/or the transformed data. In various embodiments, the one or more laboratory instruments comprises a DNA sequencer or sequencing platform, a digital spatial profiler, a spatial molecular imager, a RNA expression profiler, or a combination thereof. In some embodiments, the instrument interface allows the application to perform one or more of: monitoring the one or more laboratory instruments, receiving the data from the one or more laboratory instruments, and sending operating instructions to the one or more laboratory instruments. In some embodiments, the pipeline orchestrator tool comprises a graphic user interface (GUI) and the user creates and/or edits analysis pipelines by dragging and dropping modules, from a library of modules, within the GUI. In some embodiments, the pipeline orchestrator tool allows the user to create, edit, manage, and execute branching analysis pipelines. In some embodiments, one or more modules available in the pipeline orchestrator tool comprises training a machine learning model and/or applying a machine learning model. In some embodiments, the application further comprises a software element configured to provide a user interface allowing the user to create and manage studies. In further embodiments, the application further comprises a software element configured to provide a user interface allowing the user to collaborate and share studies. In some embodiments, the visualization is a three-dimensional (3D) representation.

In another aspect, disclosed herein are non-transitory computer-readable storage media encoded with instructions executable by one or more processors to provide a spatial biology informatics application comprising: an instrument interface communicatively coupled to one or more laboratory instruments; a software element configured to receive data, directly or indirectly, from the one or more laboratory instruments, wherein the data comprises biological image data and one or more of: genomic, proteomic, metabolomic, metagenomic, phenomic, and transcriptomic data; a software element configured to provide a pipeline orchestrator tool allowing a user to create, edit, manage, and execute analysis pipelines, wherein the pipeline orchestrator tool allows the user to link together modules and run them subsequently or in parallel to transform the received data; and a software element configured to generate a visualization of the received and/or the transformed data.

In various embodiments, the one or more laboratory instruments comprises a DNA sequencer or sequencing platform, a digital spatial profiler, a spatial molecular imager, a RNA expression profiler, or a combination thereof. In some embodiments, the instrument interface allows the application to perform one or more of: monitoring the one or more laboratory instruments, receiving the data from the one or more laboratory instruments, and sending operating instructions to the one or more laboratory instruments. In some embodiments, the pipeline orchestrator tool comprises a graphic user interface (GUI) and the user creates and/or edits analysis pipelines by dragging and dropping modules, from a library of modules, within the GUI. In some embodiments, the pipeline orchestrator tool allows the user to create, edit, manage, and execute branching analysis pipelines. In some embodiments, one or more modules available in the pipeline orchestrator tool comprises training a machine learning model and/or applying a machine learning model. In some embodiments, the application further comprises a software element configured to provide a user interface allowing the user to create and manage studies. In further embodiments, the application further comprises a software element configured to provide a user interface allowing the user to collaborate and share studies. In some embodiments, the visualization is a three-dimensional (3D) representation.

In another aspect, disclosed herein are computer-implemented methods comprising: providing, at a computer, an instrument interface communicatively coupled to one or more laboratory instruments; receiving, at the instrument interface, data from the one or more laboratory instruments, wherein the data comprises biological image data and one or more of: genomic, proteomic, metabolomic, metagenomic, phenomic, and transcriptomic data; providing, at the computer, a pipeline orchestrator tool allowing a user to create, edit, manage, and execute analysis pipelines, wherein the pipeline orchestrator tool allows the user to link together modules and run them subsequently or in parallel to transform the received data; and generating a visualization, by the computer, of the received and/or the transformed data. In various embodiments, the one or more laboratory instruments comprises a DNA sequencer or sequencing platform, a digital spatial profiler, a spatial molecular imager, a RNA expression profiler, or a combination thereof. In some embodiments, the instrument interface allows further performance of one or more of: monitoring the one or more laboratory instruments, receiving the data from the one or more laboratory instruments, and sending operating instructions to the one or more laboratory instruments. In some embodiments, the pipeline orchestrator tool comprises a graphic user interface (GUI) and the user creates and/or edits analysis pipelines by dragging and dropping modules, from a library of modules, within the GUI. In some embodiments, the pipeline orchestrator tool allows the user to create, edit, manage, and execute branching analysis pipelines. In some embodiments, one or more modules available in the pipeline orchestrator tool comprises training a machine learning model and/or applying a machine learning model. In some embodiments, the method further comprises providing a user interface allowing the user to create and manage studies. In further embodiments, the method further comprises providing a user interface allowing the user to collaborate and share studies. In some embodiments, the visualization is a three-dimensional (3D) representation.

In various embodiments, the platforms, systems, media, and methods disclosed herein include features and functionality for image analysis and storage as well as data analysis, data visualization, artificial intelligence (AI) and machine learning (ML) support, global collaboration, and scalable compute and storage capacity.

In some embodiments, the platforms, systems, media, and methods disclosed herein integrate with biology/biochemistry laboratory equipment such as nucleic acid sequencers, digital spatial profilers (such as GeoMx®), spatial multi-omics single-cell imaging platforms (such as CosMx™) and/or RNA expression profilers (such as nCounter®).

The GeoMx® Digital Spatial Profiler (DSP) is a platform for capturing spatially resolved high-plex gene (or protein) expression data from tissue. In some embodiments, DSP comprises function of RNA assays to profile the whole transcriptome from tissues on a single formalin-fixed paraffin-embedded (FFPE) sample or fresh frozen (FF) sample slide. In some embodiments, DSP comprises function of protein assays to generate quantitative and spatial analysis of multiple proteins from a single FFPE or FF sample slide. In some embodiments, FFPE or FF tissue sections may be stained with barcoded in-situ hybridization probes that bind to endogenous mRNA transcripts. In some further embodiments, a user may select regions of the interest (ROI) to profile; if desired, each ROI segment can be further sub-divided into areas of illumination (AOI) based on tissue morphology. In some further embodiments, the GeoMx® may photo-cleave and collect expression tags or barcodes for each AOI segment separately. In some further embodiments, the tags or barcodes may be used for downstream sequencing and data processing. Workflows with digital spatial profilers, in some embodiments, comprise, for example, image slide, select regions of interest (ROIs), collect ROIs, sequence, data processing, QC and normalization, and data visualization and interpretation.

The CosMx™ spatial multi-omics imager (SMI) platform is an integrated system with mature cyclic fluorescent in situ hybridization (FISH) chemistry, high-resolution imaging readout, interactive data analysis and visualization software. Workflows with CosMx™ SMI, in some embodiments, comprise, for example, sample preparation, integrated readout, or interactive data analysis. In some embodiments, sample preparation may comprise permeabilization, or fixation of the targets. In some further embodiments, sample preparation may comprise hybridization to allow RNA specific probes or antibodies binding to the targets. In some further embodiments, sample preparation comprises flow cell assembly. In some embodiments, workflow comprises multiple cycles of hybridization, imaging with UV cleavage or fluorescent dye washes.

In various embodiments, the data analysis comprises: 1) primary data analysis, e.g., the machine specific steps needed to call base pairs and compute quality scores for those calls, 2) secondary data analysis, referred to as a “pipeline,” e.g., alignment and assembly of DNA or RNA fragments providing the full sequence for a sample, from which genetic variants can be determined, and/or 3) tertiary data analysis, e.g., from sequence data, using biological data mining and interpretation tools to convert data into knowledge.

Primary data on various laboratory instruments may comprise different formats, therefore primary data analysis on laboratory instruments may comprise decoding of the primary data to correspond with the presence of a particular target identity. In some embodiments, primary data on CosMx™ SMI may comprise a series of fluorescent signals of a limited number of colors, which are detected at particular times in the instrument cycles. In some embodiments, primary data on CosMx™ SMI may comprise a single detected color at a particular cycle for the presence of a particular target. In some embodiments, primary data on CosMx™ SMI may comprise a series of colors at particular cycle for the presence of a particular target. In some embodiments, primary data on nCounter® instrument may comprise a linear sequence of fluorescent molecules of a limited number of colors identifying a particular target based on the color sequence. In some embodiments, the primary data on GeoMx® DSP may comprise a format as an nCounter instrument readout. In some embodiments, the primary data of GeoMx® DSP may comprise a format as a NGS (next generation sequencer) readout. In some embodiments, primary data in format of NGS readout on GeoMx® DSP may be further linked to a particular target RNA, DNA or protein molecule in the analysis stage.

In various embodiments, image segmentation software based on machine learning (ML) algorithms may be applied to create cell boundaries from fluorescent images of protein assays. In some embodiments, the protein assays may comprise protein antibodies binding to membrane proteins. In some embodiments, ML algorithms applied to image segmentation may comprise semantic segmentation, instance segmentation, or generative networks for segmentation. In some embodiments, image segmentation software may comprise ImageJ, CellProfiller, Cellpose, Ilastik, or QuPath.

In various embodiments, applications and use cases include, by way of non-limiting examples, discover and map cell types and cell states, phenotype of tissue microenvironment, differential expression of cell type based on spatial context, quantify subcellular expression, and spatially resolved biomarker identification.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

A better understanding of the features and advantages of the present subject matter will be obtained by reference to the following detailed description that sets forth illustrative embodiments and the accompanying drawings of which:

FIG. 1 shows a non-limiting example of a computing device; in this case, a device with one or more processors, memory, storage, and a network interface;

FIG. 2 shows a non-limiting example of a web/mobile application provision system; in this case, a system providing browser-based and/or native mobile user interfaces;

FIG. 3 shows a non-limiting example of a cloud-based web/mobile application provision system; in this case, a system comprising an elastically load balanced, auto-scaling web server and application server resources as well synchronously replicated databases;

FIG. 4 shows a non-limiting example of a graphic user interface (GUI) for a spatial biology informatics integration portal; in this case, a GUI including a default study screen;

FIG. 5 shows a non-limiting example of a GUI for a spatial biology informatics integration portal; in this case, a GUI including a pipeline orchestrator tool allowing a user to create and edit analysis pipelines by dragging-and-dropping pre-defined, editable module elements from a toolbox and linking them together;

FIG. 6 shows a non-limiting example of a GUI for a spatial biology informatics integration portal; in this case, a GUI including a pipeline editing environment for a pipeline orchestrator allowing a user to edit pipelines and/or module run parameters;

FIG. 7 shows a non-limiting example of a GUI for a spatial biology informatics integration portal; in this case, a GUI including a pipeline branching feature for a pipeline orchestrator allowing a user to perform iterative analysis by modifying parameters and re-running a step or creating a new branch of the same pipeline;

FIG. 8 shows a non-limiting example of a GUI for a spatial biology informatics integration portal; in this case, a GUI including an environment for opening existing studies;

FIG. 9 shows a non-limiting example of a GUI for a spatial biology informatics integration portal; in this case, a GUI including an environment for viewing an analysis pipeline for a study as well as visualizing results of a module of the pipeline;

FIG. 10 shows a non-limiting example of a GUI for a spatial biology informatics integration portal; in this case, a GUI including customizable layout options, such as, variable sizing of different windowpanes;

FIGS. 11A-11C show a non-limiting example of a GUI for a spatial biology informatics integration portal; in this case, a GUI including an environment to graphically represent data as X-Y scatterplots of, for example, cell or transcript coordinates;

FIGS. 12A-12C show a non-limiting example of a GUI for a spatial biology informatics integration portal; in this case, a GUI including an environment to graphically represent data as heatmaps;

FIG. 13 shows a non-limiting example of a GUI for a spatial biology informatics integration portal; in this case, a GUI including an environment to graphically represent data as histograms;

FIGS. 14A-14C show a non-limiting example of a GUI for a spatial biology informatics integration portal; in this case, a GUI including an environment to graphically represent data as boxplots;

FIG. 15 shows a non-limiting example of a GUI for a spatial biology informatics integration portal; in this case, a GUI including an environment to graphically represent data as violin plots;

FIGS. 16A and 16B show a non-limiting example of a GUI for a spatial biology informatics integration portal; in this case, a GUI including an environment to perform dimension reduction including by, for example, Principal Component Analysis (PCA) and Uniform Manifold Approximation and Projection (UMAP);

FIGS. 17A and 17B shows a non-limiting example of a GUI for a spatial biology informatics integration portal; in this case, a GUI including an environment allowing a user to annotate data by using drawing tools to identify regions of images and/or graphical plots;

FIG. 18 shows a non-limiting example of a GUI for a spatial biology informatics integration portal; in this case, a GUI including a test annotation service for interactive annotations linked to a flow cell image and table;

FIGS. 19-22 show a non-limiting example of a GUI for a spatial biology informatics integration portal; in this case, a GUI including an environment for interactive data viewing allowing a user to select a flow cell image and visualize relevant data using any running pipeline module;

FIG. 23 shows a non-limiting example of a GUI for a spatial biology informatics integration portal; in this case, a GUI including an environment for data visualization with image viewer integration allowing a user to control the image viewer display and see updates to relevant visualized data;

FIGS. 24-30, 31A, 31B, 32, 33A, 33B, 34A and 34B show exemplary data from a spatial molecular imager study of non-small cell lung cancer (NSCLC) formalin fixed paraffin embedded (FFPE) samples (spatially resolved high-plex RNA data); in this case, a study in which the subject matter described herein was used to investigate ecosystems existing within the tissues, how the cells respond to their environments, and how cells interact with their neighbors;

FIG. 35 shows a non-limiting example of an architecture diagram; in this case, an architecture diagram illustrating data access patterns;

FIGS. 36-40 show a first non-limiting exemplary embodiment of a spatial biology informatics integration portal;

FIGS. 41-61 show a second non-limiting exemplary embodiment of a spatial biology informatics integration portal;

FIG. 62 shows a non-limiting example of Pipeline Orchestrator Interaction Editing interface;

FIG. 63 shows a non-limiting example of User Interface enhancement for quality control (QC);

FIG. 64 shows a non-limiting example of Cell Segmentation Module Illustration;

FIG. 65 shows a non-limiting example of cell segmentation overlay when launching image viewer;

FIG. 66 shows a non-limiting example of a display from Cell Typing module;

FIG. 67 shows a non-limiting example of overview of the application architecture and services for development of graphic processing;

FIG. 68 shows a non-limiting example of a RNA transcript plot;

FIG. 69 shows a non-limiting example of Uniform Manifold Approximation and Projection (UMAP);

FIG. 70 shows a non-limiting example of graph of in situ RNA cell typing;

FIG. 71 shows a non-limiting example of graph of Leiden Clustering;

FIG. 72 shows a non-limiting example of graph display of Spatial Expression Analysis;

FIG. 73 shows a non-limiting example of heatmap from Cell Type Co-localization analysis;

FIG. 74 shows a non-limiting example of graph display of Marker Genes module analysis; and

FIG. 75 shows a non-limiting example of graph display of Ligand-Receptor analysis.

### DETAILED DESCRIPTION

Described herein, in certain embodiments, are systems comprising at least one processor and instructions executable by the at least one processor to provide a spatial biology informatics application comprising: an instrument interface communicatively coupled to one or more laboratory instruments; a software element configured to receive data, directly or indirectly, from the one or more laboratory instruments, wherein the data comprises biological image data and one or more of: genomic, proteomic, metabolomic, metagenomic, phenomic, and transcriptomic data; a software element configured to provide a pipeline orchestrator tool allowing a user to create, edit, manage, and execute analysis pipelines, wherein the pipeline orchestrator tool allows the user to link together modules and run them subsequently or in parallel to transform the received data; and a software element configured to generate a visualization of the received and/or the transformed data. In various embodiments, the one or more laboratory instruments comprises a DNA sequencer or sequencing platform, a digital spatial profiler, a spatial molecular imager, a RNA expression profiler, or a combination thereof. In some embodiments, the instrument interface allows the application to perform one or more of: monitoring the one or more laboratory instruments, receiving the data from the one or more laboratory instruments, and sending operating instructions to the one or more laboratory instruments. In some embodiments, the pipeline orchestrator tool comprises a graphic user interface (GUI) and the user creates and/or edits analysis pipelines by dragging and dropping modules, from a library of modules, within the GUI. In some embodiments, the pipeline orchestrator tool allows the user to create, edit, manage, and execute branching analysis pipelines. In some embodiments, one or more modules available in the pipeline orchestrator tool comprises training a machine learning model and/or applying a machine learning model. In some embodiments, the application further comprises a software element configured to provide a user interface allowing the user to create and manage studies. In further embodiments, the application further comprises a software element configured to provide a user interface allowing the user to collaborate and share studies. In some embodiments, the visualization is a three-dimensional (3D) representation.

Also described herein, in certain embodiments, are non-transitory computer-readable storage media encoded with instructions executable by one or more processors to provide a spatial biology informatics application comprising: an instrument interface communicatively coupled to one or more laboratory instruments; a software element configured to receive data, directly or indirectly, from the one or more laboratory instruments, wherein the data comprises biological image data and one or more of: genomic, proteomic, metabolomic, metagenomic, phenomic, and transcriptomic data; a software element configured to provide a pipeline orchestrator tool allowing a user to create, edit, manage, and execute analysis pipelines, wherein the pipeline orchestrator tool allows the user to link together modules and run them subsequently or in parallel to transform the received data; and a software element configured to generate a visualization of the received and/or the transformed data. In various embodiments, the one or more laboratory instruments comprises a DNA sequencer or sequencing platform, a digital spatial profiler, a spatial molecular imager, a RNA expression profiler, or a combination thereof. In some embodiments, the instrument interface allows the application to perform one or more of: monitoring the one or more laboratory instruments, receiving the data from the one or more laboratory instruments, and sending operating instructions to the one or more laboratory instruments. In some embodiments, the pipeline orchestrator tool comprises a graphic user interface (GUI) and the user creates and/or edits analysis pipelines by dragging and dropping modules, from a library of modules, within the GUI. In some embodiments, the pipeline orchestrator tool allows the user to create, edit, manage, and execute branching analysis pipelines. In some embodiments, one or more modules available in the pipeline orchestrator tool comprises training a machine learning model and/or applying a machine learning model. In some embodiments, the application further comprises a software element configured to provide a user interface allowing the user to create and manage studies. In further embodiments, the application further comprises a software element configured to provide a user interface allowing the user to collaborate and share studies. In some embodiments, the visualization is a three-dimensional (3D) representation.

Also described herein, in certain embodiments, are computer-implemented methods comprising: providing, at a computer, an instrument interface communicatively coupled to one or more laboratory instruments; receiving, at the instrument interface, data from the one or more laboratory instruments, wherein the data comprises biological image data and one or more of: genomic, proteomic, metabolomic, metagenomic, phenomic, and transcriptomic data; providing, at the computer, a pipeline orchestrator tool allowing a user to create, edit, manage, and execute analysis pipelines, wherein the pipeline orchestrator tool allows the user to link together modules and run them subsequently or in parallel to transform the received data; and generating a visualization, by the computer, of the received and/or the transformed data. In various embodiments, the one or more laboratory instruments comprises a DNA sequencer or sequencing platform, a digital spatial profiler, a spatial molecular imager, a RNA expression profiler, or a combination thereof. In some embodiments, the instrument interface allows further performance of one or more of: monitoring the one or more laboratory instruments, receiving the data from the one or more laboratory instruments, and sending operating instructions to the one or more laboratory instruments. In some embodiments, the pipeline orchestrator tool comprises a graphic user interface (GUI) and the user creates and/or edits analysis pipelines by dragging and dropping modules, from a library of modules, within the GUI. In some embodiments, the pipeline orchestrator tool allows the user to create, edit, manage, and execute branching analysis pipelines. In some embodiments, one or more modules available in the pipeline orchestrator tool comprises training a machine learning model and/or applying a machine learning model. In some embodiments, the method further comprises providing a user interface allowing the user to create and manage studies. In further embodiments, the method further comprises providing a user interface allowing the user to collaborate and share studies. In some embodiments, the visualization is a three-dimensional (3D) representation.

### Certain Definitions

Unless otherwise defined, all technical terms used herein have the same meaning as commonly understood by one of ordinary skill in the art to which the present subject matter belongs.

As used in this specification and the appended claims, the singular forms “a,” “an,” and “the” include plural references unless the context clearly dictates otherwise. Any reference to “or” herein is intended to encompass “and/or” unless otherwise stated.

Reference throughout this specification to “some embodiments,” “further embodiments,” or “a particular embodiment,” means that a particular feature, structure, or characteristic described in connection with the embodiment is included in at least one embodiment. Thus, the appearances of the phrase “in some embodiments,” or “in further embodiments,” or “in a particular embodiment” in various places throughout this specification are not necessarily all referring to the same embodiment. Furthermore, the particular features, structures, or characteristics may be combined in any suitable manner in one or more embodiments.

### Computing System

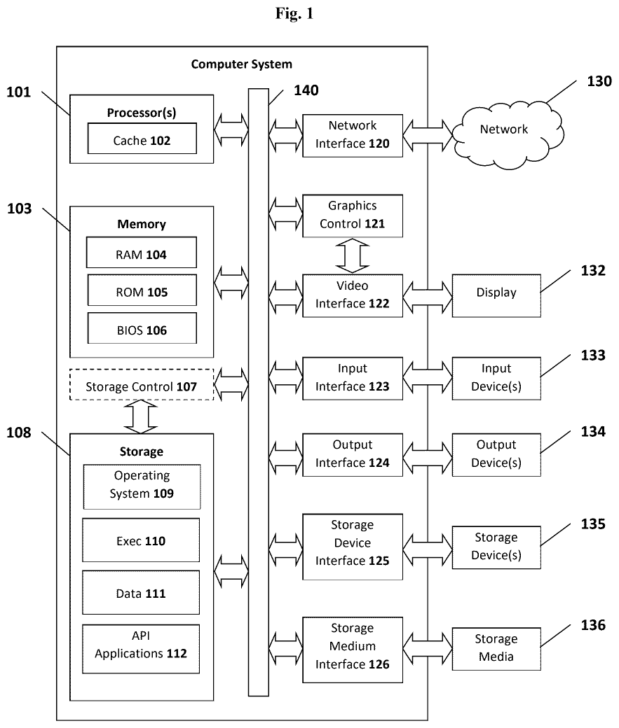

Referring to FIG. 1, a block diagram is shown depicting an exemplary machine that includes a computer system 100 (e.g., a processing or computing system) within which a set of instructions can execute for causing a device to perform or execute any one or more of the aspects and/or methodologies for static code scheduling of the present disclosure. The components in are examples only and do not limit the scope of use or functionality of any hardware, software, embedded logic component, or a combination of two or more such components implementing particular embodiments.

Computer system 100 may include one or more processors 101, a memory 103, and a storage 108 that communicate with each other, and with other components, via a bus 140. The bus 140 may also link a display 132, one or more input devices 133 (which may, for example, include a keypad, a keyboard, a mouse, a stylus, etc.), one or more output devices 134, one or more storage devices 135, and various tangible storage media 136. All of these elements may interface directly or via one or more interfaces or adaptors to the bus 140. For instance, the various tangible storage media 136 can interface with the bus 140 via storage medium interface 126. Computer system 100 may have any suitable physical form, including but not limited to one or more integrated circuits (ICs), printed circuit boards (PCBs), mobile handheld devices (such as mobile telephones or PDAs), laptop or notebook computers, distributed computer systems, computing grids, or servers.

Computer system 100 includes one or more processor(s) 101 (e.g., central processing units (CPUs), general purpose graphics processing units (GPGPUs), or quantum processing units (QPUs)) that carry out functions. Processor(s) 101 optionally contains a cache memory unit 102 for temporary local storage of instructions, data, or computer addresses. Processor(s) 101 are configured to assist in execution of computer readable instructions. Computer system 100 may provide functionality for the components depicted in FIG. 1 as a result of the processor(s) 101 executing non-transitory, processor-executable instructions embodied in one or more tangible computer-readable storage media, such as memory 103, storage 108, storage devices 135, and/or storage medium 136. The computer-readable media may store software that implements particular embodiments, and processor(s) 101 may execute the software. Memory 103 may read the software from one or more other computer-readable media (such as mass storage device(s) 135, 136) or from one or more other sources through a suitable interface, such as network interface 120. The software may cause processor(s) 101 to carry out one or more processes or one or more steps of one or more processes described or illustrated herein. Carrying out such processes or steps may include defining data structures stored in memory 103 and modifying the data structures as directed by the software.

The memory 103 may include various components (e.g., machine readable media) including, but not limited to, a random access memory component (e.g., RAM 104) (e.g., static RAM (SRAM), dynamic RAM (DRAM), ferroelectric random access memory (FRAM), phase-change random access memory (PRAM), etc.), a read-only memory component (e.g., ROM 105), and any combinations thereof. ROM 105 may act to communicate data and instructions unidirectionally to processor(s) 101, and RAM 104 may act to communicate data and instructions bidirectionally with processor(s) 101. ROM 105 and RAM 104 may include any suitable tangible computer-readable media described below. In one example, a basic input/output system 106 (BIOS), including basic routines that help to transfer information between elements within computer system 100, such as during start-up, may be stored in the memory 103.

Fixed storage 108 is connected bidirectionally to processor(s) 101, optionally through storage control unit 107. Fixed storage 108 provides additional data storage capacity and may also include any suitable tangible computer-readable media described herein. Storage 108 may be used to store operating system 109, executable(s) 110, data 111, applications 112 (application programs), and the like. Storage 108 can also include an optical disk drive, a solid-state memory device (e.g., flash-based systems), or a combination of any of the above. Information in storage 108 may, in appropriate cases, be incorporated as virtual memory in memory 103.

In one example, storage device(s) 135 may be removably interfaced with computer system 100 (e.g., via an external port connector (not shown)) via a storage device interface 125. Particularly, storage device(s) 135 and an associated machine-readable medium may provide non-volatile and/or volatile storage of machine-readable instructions, data structures, program modules, and/or other data for the computer system 100. In one example, software may reside, completely or partially, within a machine-readable medium on storage device(s) 135. In another example, software may reside, completely or partially, within processor(s) 101.

Bus 140 connects a wide variety of subsystems. Herein, reference to a bus may encompass one or more digital signal lines serving a common function, where appropriate. Bus 140 may be any of several types of bus structures including, but not limited to, a memory bus, a memory controller, a peripheral bus, a local bus, and any combinations thereof, using any of a variety of bus architectures. As an example and not by way of limitation, such architectures include an Industry Standard Architecture (ISA) bus, an Enhanced ISA (EISA) bus, a Micro Channel Architecture (MCA) bus, a Video Electronics Standards Association local bus (VLB), a Peripheral Component Interconnect (PCI) bus, a PCI-Express (PCI-X) bus, an Accelerated Graphics Port (AGP) bus, HyperTransport (HTX) bus, serial advanced technology attachment (SATA) bus, and any combinations thereof.

Computer system 100 may also include an input device 133. In one example, a user of computer system 100 may enter commands and/or other information into computer system 100 via input device(s) 133. Examples of an input device(s) 133 include, but are not limited to, an alpha-numeric input device (e.g., a keyboard), a pointing device (e.g., a mouse or touchpad), a touchpad, a touch screen, a multi-touch screen, a joystick, a stylus, a gamepad, an audio input device (e.g., a microphone, a voice response system, etc.), an optical scanner, a video or still image capture device (e.g., a camera), and any combinations thereof. In some embodiments, the input device is a Kinect, Leap Motion, or the like. Input device(s) 133 may be interfaced to bus 140 via any of a variety of input interfaces 123 (e.g., input interface 123) including, but not limited to, serial, parallel, game port, USB, FIREWIRE, THUNDERBOLT, or any combination of the above.

In particular embodiments, when computer system 100 is connected to network 130, computer system 100 may communicate with other devices, specifically mobile devices and enterprise systems, distributed computing systems, cloud storage systems, cloud computing systems, and the like, connected to network 130. Communications to and from computer system 100 may be sent through network interface 120. For example, network interface 120 may receive incoming communications (such as requests or responses from other devices) in the form of one or more packets (such as Internet Protocol (IP) packets) from network 130, and computer system 100 may store the incoming communications in memory 103 for processing. Computer system 100 may similarly store outgoing communications (such as requests or responses to other devices) in the form of one or more packets in memory 103 and communicated to network 130 from network interface 120. Processor(s) 101 may access these communication packets stored in memory 103 for processing.

Examples of the network interface 120 include, but are not limited to, a network interface card, a modem, and any combination thereof. Examples of a network 130 or network segment 130 include, but are not limited to, a distributed computing system, a cloud computing system, a wide area network (WAN) (e.g., the Internet, an enterprise network), a local area network (LAN) (e.g., a network associated with an office, a building, a campus or other relatively small geographic space), a telephone network, a direct connection between two computing devices, a peer-to-peer network, and any combinations thereof. A network, such as network 130, may employ a wired and/or a wireless mode of communication. In general, any network topology may be used.

Information and data can be displayed through a display 132. Examples of a display 132 include, but are not limited to, a cathode ray tube (CRT), a liquid crystal display (LCD), a thin film transistor liquid crystal display (TFT-LCD), an organic liquid crystal display (OLED) such as a passive-matrix OLED (PMOLED) or active-matrix OLED (AMOLED) display, a plasma display, and any combinations thereof. The display 132 can interface to the processor(s) 101, memory 103, and fixed storage 108, as well as other devices, such as input device(s) 133, via the bus 140. The display 132 is linked to the bus 140 via a video interface 122, and transport of data between the display 132 and the bus 140 can be controlled via the graphics control 121. In some embodiments, the display is a video projector. In some embodiments, the display is a head-mounted display (HMD) such as a VR headset. In further embodiments, suitable VR headsets include, by way of non-limiting examples, HTC Vive, Oculus Rift, Samsung Gear VR, Microsoft HoloLens, Razer OSVR, FOVE VR, Zeiss VR One, Avegant Glyph, Freefly VR headset, and the like. In still further embodiments, the display is a combination of devices such as those disclosed herein.

In addition to a display 132, computer system 100 may include one or more other peripheral output devices 134 including, but not limited to, an audio speaker, a printer, a storage device, and any combinations thereof. Such peripheral output devices may be connected to the bus 140 via an output interface 124. Examples of an output interface 124 include, but are not limited to, a serial port, a parallel connection, a USB port, a FIREWIRE port, a THUNDERBOLT port, and any combinations thereof.

In addition or as an alternative, computer system 100 may provide functionality as a result of logic hardwired or otherwise embodied in a circuit, which may operate in place of or together with software to execute one or more processes or one or more steps of one or more processes described or illustrated herein. Reference to software in this disclosure may encompass logic, and reference to logic may encompass software. Moreover, reference to a computer-readable medium may encompass a circuit (such as an IC) storing software for execution, a circuit embodying logic for execution, or both, where appropriate. The present disclosure encompasses any suitable combination of hardware, software, or both.

Those of skill in the art will appreciate that the various illustrative logical blocks, modules, circuits, and algorithm steps described in connection with the embodiments disclosed herein may be implemented as electronic hardware, computer software, or combinations of both. To clearly illustrate this interchangeability of hardware and software, various illustrative components, blocks, modules, circuits, and steps have been described above generally in terms of their functionality.

The various illustrative logical blocks, modules, and circuits described in connection with the embodiments disclosed herein may be implemented or performed with a general purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general purpose processor may be a microprocessor, but in the alternative, the processor may be any conventional processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration.

The steps of a method or algorithm described in connection with the embodiments disclosed herein may be embodied directly in hardware, in a software module executed by one or more processor(s), or in a combination of the two. A software module may reside in RAM memory, flash memory, ROM memory, EPROM memory, EEPROM memory, registers, hard disk, a removable disk, a CD-ROM, or any other form of storage medium known in the art. An exemplary storage medium is coupled to the processor such the processor can read information from, and write information to, the storage medium. In the alternative, the storage medium may be integral to the processor. The processor and the storage medium may reside in an ASIC. The ASIC may reside in a user terminal. In the alternative, the processor and the storage medium may reside as discrete components in a user terminal.

In accordance with the description herein, suitable computing devices include, by way of non-limiting examples, distributed computing systems, cloud computing platforms, server clusters, server computers, desktop computers, laptop computers, notebook computers, sub-notebook computers, netbook computers, netpad computers, handheld computers, Internet appliances, mobile smartphones, and tablet computers. Those of skill in the art will also recognize that select televisions, video players, and digital music players with optional computer network connectivity are suitable for use in the system described herein. Suitable tablet computers, in various embodiments, include those with booklet, slate, and convertible configurations, known to those of skill in the art.

In some embodiments, the computing device includes an operating system configured to perform executable instructions. The operating system is, for example, software, including programs and data, which manages the device's hardware and provides services for execution of applications. Those of skill in the art will recognize that suitable server operating systems include, by way of non-limiting examples, FreeBSD, OpenBSD, NetBSD®, Linux, Apple® Mac OS X Server®, Oracle® Solaris®, Windows Server®, and Novell® NetWare®. Those of skill in the art will recognize that suitable personal computer operating systems include, by way of non-limiting examples, Microsoft® Windows®, Apple® Mac OS X®, UNIX®, and UNIX-like operating systems such as GNU/Linux®. In some embodiments, the operating system is provided by cloud computing. Those of skill in the art will also recognize that suitable mobile smartphone operating systems include, by way of non-limiting examples, Nokia® Symbian® OS, Apple® iOS®, Research In Motion® BlackBerry OS®, Google® Android®, Microsoft® Windows Phone® OS, Microsoft® Windows Mobile® OS, Linux®, and Palm® WebOS®.

### Non-Transitory Computer Readable Storage Medium

In some embodiments, the platforms, systems, media, and methods disclosed herein include one or more non-transitory computer readable storage media encoded with a program including instructions executable by the operating system of an optionally networked computing device. In further embodiments, a computer readable storage medium is a tangible component of a computing device. In still further embodiments, a computer readable storage medium is optionally removable from a computing device. In some embodiments, a computer readable storage medium includes, by way of non-limiting examples, CD-ROMs, DVDs, flash memory devices, solid state memory, magnetic disk drives, magnetic tape drives, optical disk drives, distributed computing systems including cloud computing systems and services, and the like. In some cases, the program and instructions are permanently, substantially permanently, semi-permanently, or non-transitorily encoded on the media.

### Computer Program

In some embodiments, the platforms, systems, media, and methods disclosed herein include at least one computer program, or use of the same. A computer program includes a sequence of instructions, executable by one or more processor(s) of the computing device's CPU, written to perform a specified task. Computer readable instructions may be implemented as program modules, such as functions, objects, Application Programming Interfaces (APIs), computing data structures, and the like, that perform particular tasks or implement particular abstract data types. In light of the disclosure provided herein, those of skill in the art will recognize that a computer program may be written in various versions of various languages.

The functionality of the computer readable instructions may be combined or distributed as desired in various environments. In some embodiments, a computer program comprises one sequence of instructions. In some embodiments, a computer program comprises a plurality of sequences of instructions. In some embodiments, a computer program is provided from one location. In other embodiments, a computer program is provided from a plurality of locations. In various embodiments, a computer program includes one or more software modules. In various embodiments, a computer program includes, in part or in whole, one or more web applications, one or more mobile applications, one or more standalone applications, one or more web browser plug-ins, extensions, add-ins, or add-ons, or combinations thereof.

### Web Application

In some embodiments, a computer program includes a web application. In light of the disclosure provided herein, those of skill in the art will recognize that a web application, in various embodiments, utilizes one or more software frameworks and one or more database systems. In some embodiments, a web application is created upon a software framework such as Microsoft®.NET or Ruby on Rails (RoR). In some embodiments, a web application utilizes one or more database systems including, by way of non-limiting examples, relational, non-relational, object oriented, associative, XML, and document oriented database systems. In further embodiments, suitable relational database systems include, by way of non-limiting examples, Microsoft® SQL Server, mySQL™, and Oracle®. Those of skill in the art will also recognize that a web application, in various embodiments, is written in one or more versions of one or more languages. A web application may be written in one or more markup languages, presentation definition languages, client-side scripting languages, server-side coding languages, database query languages, or combinations thereof. In some embodiments, a web application is written to some extent in a markup language such as Hypertext Markup Language (HTML), Extensible Hypertext Markup Language (XHTML), or eXtensible Markup Language (XML). In some embodiments, a web application is written to some extent in a presentation definition language such as Cascading Style Sheets (CSS). In some embodiments, a web application is written to some extent in a client-side scripting language such as Asynchronous JavaScript and XML (AJAX), Flash® ActionScript, JavaScript, or Silverlight®. In some embodiments, a web application is written to some extent in a server-side coding language such as Active Server Pages (ASP), ColdFusion®, Perl, Java™, JavaServer Pages (JSP), Hypertext Preprocessor (PHP), Python™, Ruby, Tcl, Smalltalk, WebDNA®, or Groovy. In some embodiments, a web application is written to some extent in a database query language such as Structured Query Language (SQL). In some embodiments, a web application integrates enterprise server products such as IBM© Lotus Domino®. In some embodiments, a web application includes a media player element. In various further embodiments, a media player element utilizes one or more of many suitable multimedia technologies including, by way of non-limiting examples, Adobe® Flash®, HTML 5, Apple® QuickTime®, Microsoft® Silverlight®, Java™, and Unity®.

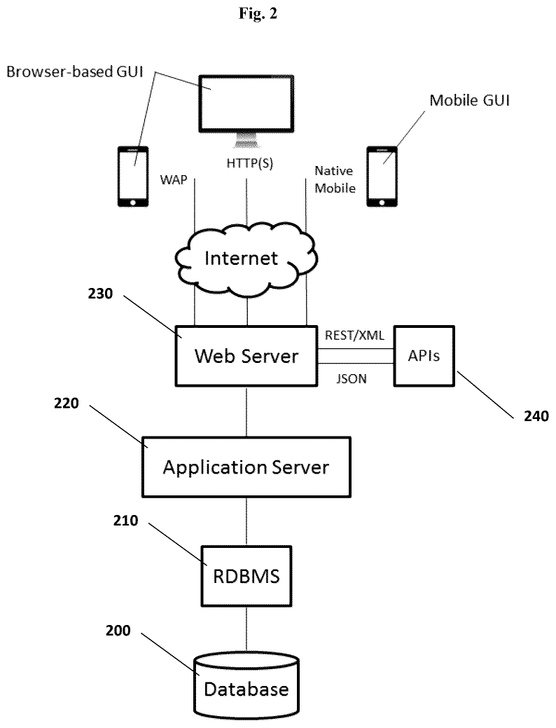

Referring to FIG. 2, in a particular embodiment, an application provision system comprises one or more databases 200 accessed by a relational database management system (RDBMS) 210. Suitable RDBMSs include Firebird, MySQL, PostgreSQL, SQLite, Oracle Database, Microsoft SQL Server, IBM DB2, IBM Informix, SAP Sybase, Teradata, and the like. In this embodiment, the application provision system further comprises one or more application severs 220 (such as Java servers, .NET servers, PHP servers, and the like) and one or more web servers 230 (such as Apache, IIS, GWS and the like). The web server(s) optionally expose one or more web services via app application programming interfaces (APIs) 240. Via a network, such as the Internet, the system provides browser-based and/or mobile native user interfaces.

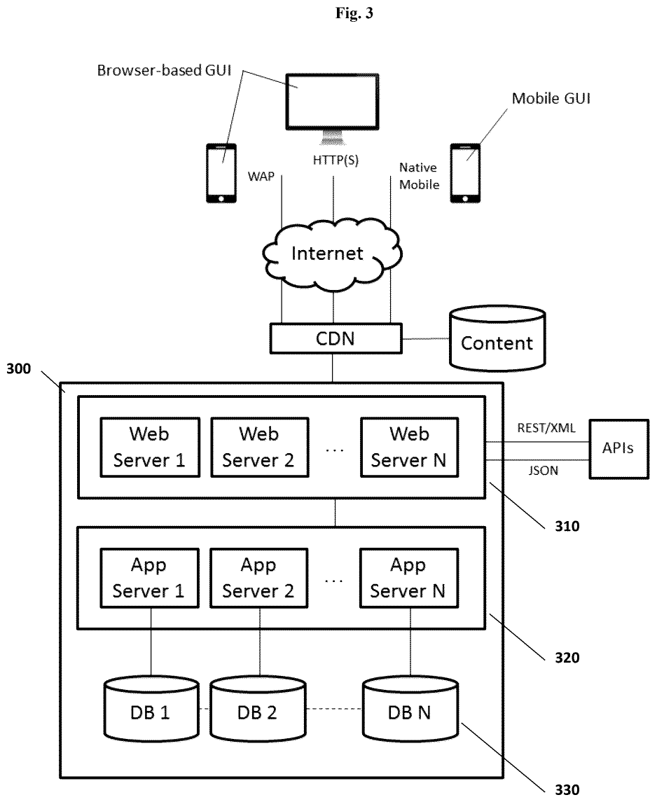

Referring to FIG. 3, in a particular embodiment, an application provision system alternatively has a distributed, cloud-based architecture 300 and comprises elastically load balanced, auto-scaling web server resources 310 and application server resources 320 as well synchronously replicated databases 330.

### Standalone Application

In some embodiments, a computer program includes a standalone application, which is a program that is run as an independent computer process, not an add-on to an existing process, e.g., not a plug-in. Those of skill in the art will recognize that standalone applications are often compiled. A compiler is a computer program(s) that transforms source code written in a programming language into binary object code such as assembly language or machine code. Suitable compiled programming languages include, by way of non-limiting examples, C, C++, Objective-C, COBOL, Delphi, Eiffel, Java™, Lisp, Python™, Visual Basic, and VB .NET, or combinations thereof. Compilation is often performed, at least in part, to create an executable program. In some embodiments, a computer program includes one or more executable complied applications.

### Software Modules

In some embodiments, the platforms, systems, media, and methods disclosed herein include software, server, and/or database modules, or use of the same. In view of the disclosure provided herein, software modules are created by techniques known to those of skill in the art using machines, software, and languages known to the art. The software modules disclosed herein are implemented in a multitude of ways. In various embodiments, a software module comprises a file, a section of code, a programming object, a programming structure, a distributed computing resource, a cloud computing resource, or combinations thereof. In further various embodiments, a software module comprises a plurality of files, a plurality of sections of code, a plurality of programming objects, a plurality of programming structures, a plurality of distributed computing resources, a plurality of cloud computing resources, or combinations thereof. In various embodiments, the one or more software modules comprise, by way of non-limiting examples, a web application, a mobile application, a standalone application, and a distributed or cloud computing application. In some embodiments, software modules are in one computer program or application. In other embodiments, software modules are in more than one computer program or application. In some embodiments, software modules are hosted on one machine. In other embodiments, software modules are hosted on more than one machine. In further embodiments, software modules are hosted on a distributed computing platform such as a cloud computing platform. In some embodiments, software modules are hosted on one or more machines in one location. In other embodiments, software modules are hosted on one or more machines in more than one location.

### Databases

In some embodiments, the platforms, systems, media, and methods disclosed herein include one or more databases, or use of the same. In view of the disclosure provided herein, those of skill in the art will recognize that many databases are suitable for storage and retrieval of user information, study information, slide information, field of view (FoV) information, flow cell information, image information, genomic information, transcriptomic information, and proteomic information. In various embodiments, suitable databases include, by way of non-limiting examples, relational databases, non-relational databases, object oriented databases, object databases, entity-relationship model databases, associative databases, XML databases, document oriented databases, and graph databases. Further non-limiting examples include SQL, PostgreSQL, MySQL, Oracle, DB2, Sybase, and MongoDB. In some embodiments, a database is Internet-based. In further embodiments, a database is web-based. In still further embodiments, a database is cloud computing-based. In a particular embodiment, a database is a distributed database. In other embodiments, a database is based on one or more local computer storage devices.

### Instrument Interface

In some embodiments, the platforms, systems, media, and methods disclosed herein include an instrument interface. In further embodiments, the instrument interface is a hardware and/or software interface communicatively coupled to one or more laboratory instruments. Many laboratory instruments are suitable, including, by way of non-limiting examples, nucleic acid sequencers or sequencing platforms, digital spatial profilers (such as the NanoString GeoMx®), a spatial molecular imager (such as the NanoString CosMx™), a RNA expression profiler (such as the NanoString nCounter® system), or a combination thereof. In some embodiments, digital spatial profilers comprise function of RNA assays to profile the whole transcriptome from tissues and/or samples on a single FFPE or fresh frozen (FF) slide. In some embodiments, digital spatial profilers comprise function of Protein assays to generate quantitative and spatial analysis of multiple proteins from a single FFPE or FF slide.

In various embodiments, the instrument interface allows the application to perform, by way of non-limiting examples, monitor one or more laboratory instruments, receive data from one or more laboratory instruments, and/or send operating instructions to one or more laboratory instruments. In some embodiments, the instrument interface is a one-way link to one or more laboratory instruments. In other embodiments, the instrument interface is a two-way link to one or more laboratory instruments.

In some embodiments, the platforms, systems, media, and methods disclosed herein receive data via an instrument interface communicatively coupled to one or more laboratory instruments. In some embodiments, the data is received directly from one or more laboratory instruments. In other embodiments, the data is received indirectly from one or more laboratory instruments. Many types of data are suitable. In the field of spatial biology, useful data includes, by way of non-limiting examples, biological image data such as microscopy images (e.g., micrographs) of formalin fixed paraffin embedded (FFPE) and/or fresh frozen (FF) samples of cells and/or tissues. In some embodiments, data from a single slide for RNA Assays and Protein Assays is split into two datasets. In some embodiments, data from a single slide for RNA assays and Protein Assays is combined. In some embodiments, the image data is two-dimensional data. In some embodiments, the image data is three-dimensional image data. Further, in the field of spatial biology, useful data includes, by way of non-limiting examples, “-omics” data such as genomic data, proteomic data, metabolomic data, metagenomic data, phenomic data, and/or transcriptomic data. In some embodiments, the -omics data is associated with the image data. In further embodiments, the -omics data is spatially associated with the image data in two and/or three-dimensions. In various embodiments, the -omics data is associated with the image data as metadata and/or as an overlay to the image. Other useful information includes, patient data, demographic data, diagnosis data, disease data, treatment data, study data, and the like.

In some embodiments, the platforms, systems, media, and methods disclosed herein may receive data via an instrument interface and keep it in its original file format. In some embodiments, the platforms, systems, media, and methods disclosed herein may receive data via an instrument interface and add it to dataset(s).

### Study Management

In some embodiments, the platforms, systems, media, and methods disclosed herein include features and functionality for study management. In further embodiments, the subject matter disclosed herein includes tools allowing a user to create a study, edit and/or modify a study, and delete a study.

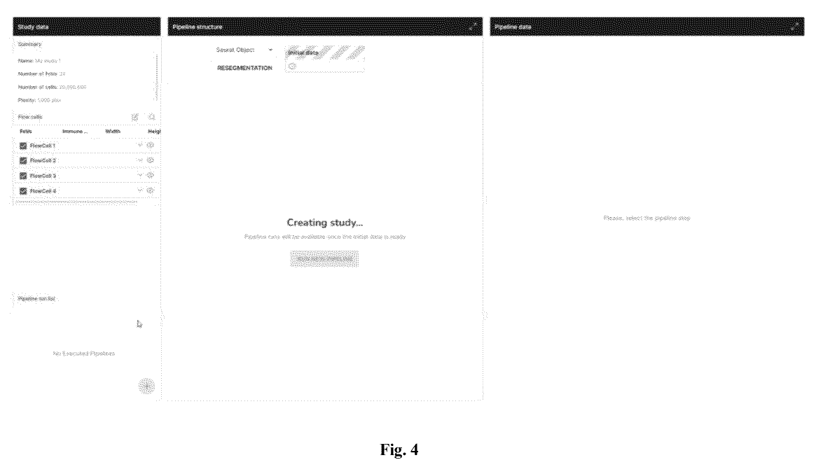

FIG. 4 shows a non-limiting example of a GUI for a spatial biology informatics integration portal including a default study screen. The study screen has a study data section, a pipeline structure section, and a pipelined data section. Continuing to refer to FIG. 4, the study data section includes study name, number of fields of view (FoVs) associated with the study, number of cells associated with the study, plexity of the study, a list of flow cells associated with the study, and a pipeline run list for the study.

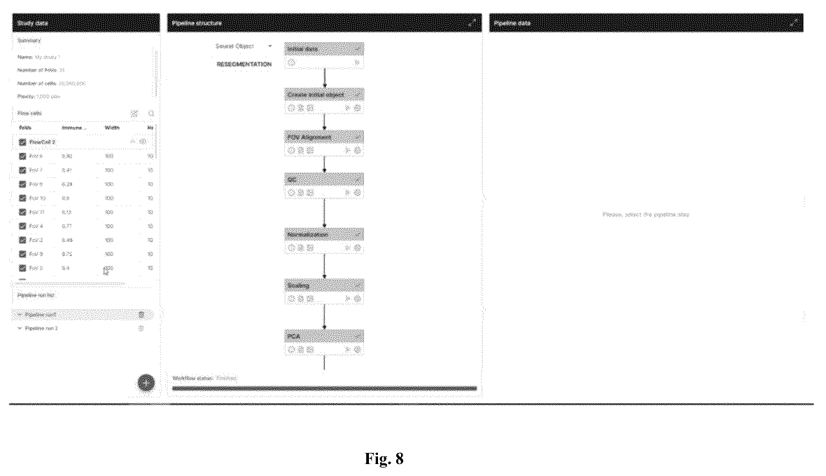

FIG. 8 shows a non-limiting example of a GUI for a spatial biology informatics integration portal including an environment for opening studies including previously created studies. Referring to FIG. 8, the pipeline structure section includes a schematic representation of a pipeline comprising a series of editable modules for a study.

### Pipeline Orchestrator

In some embodiments, the platforms, systems, media, and methods disclosed herein include a pipeline orchestrator tool. In further embodiments, a pipeline orchestrator tool includes features and functionality allowing a user to, for example, create, edit, save, open, manage, and execute analysis pipelines. In still further embodiments, a pipeline orchestrator tool includes features and functionality allowing a user to link together modules and run them subsequently or in parallel to transform data.

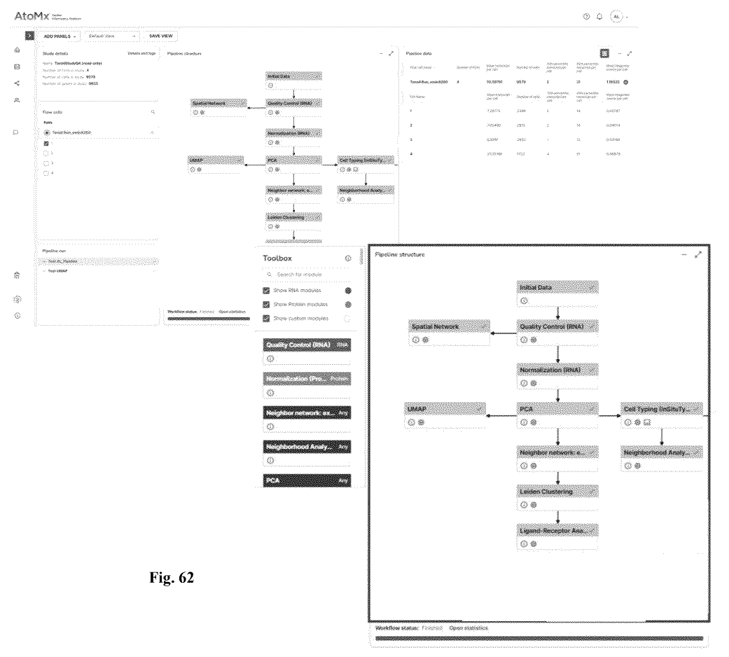

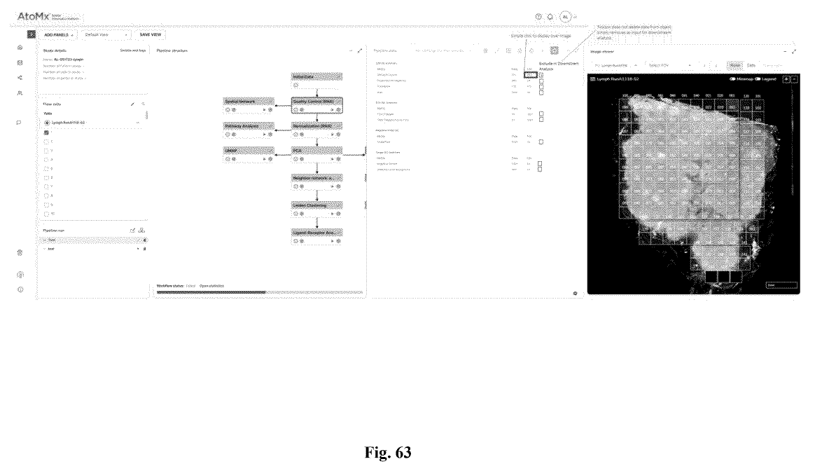

In some embodiments, a user selects modules from a library of modules. Every module may have defined “interactions” enabled for seamless integration of pipeline structure, pipeline data, image viewer, goal for every algorithm to be visualized in tissue space. FIG. 62 shows a non-limiting example of the Pipeline Orchestrator Interaction Editing module, integrating a pipeline structure pane, with full access to tool box and pipeline data. This Editing interface provides a gridded workspace to make real-time updates for end users to perform analytical tasks, with full access to tool box and modules, in combination with data displays to visualize results and adapt analysis plan. FIG. 63 shows a non-limiting example of user interface enhancement for QC. This illustrates a function connecting visualization of cells and downstream analysis selection. A simple click over the image will display the QC results. User may exclude data from entering downstream analysis based on the low QC results. However, checking tick box does not delete data from the object, but simply removes it as input for downstream analysis. In some embodiments, modules may comprise function to alter input data. In some embodiments, modules may comprise function to alter input data prior downstream module execution.

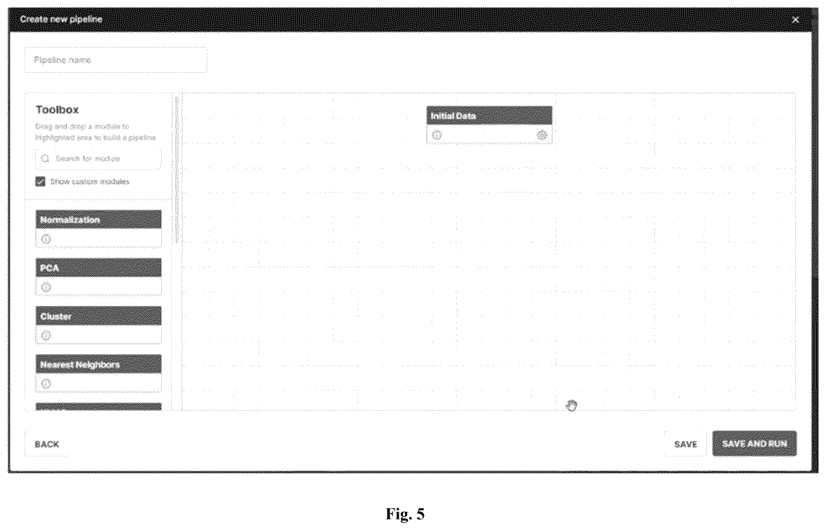

FIG. 5 shows a non-limiting example of a GUI for a spatial biology informatics integration portal including a pipeline orchestrator tool allowing a user to create and edit analysis pipelines by dragging-and-dropping pre-defined, editable module elements from a searchable toolbox and linking them together. Referring to FIG. 5, a searchable toolbox comprises a list of available modules including, normalization, PCA, cluster, nearest neighbors, and the like. The user has placed an initial data module into the interface and can name the pipeline, save the pipeline, and/or save and run the pipeline under development.

In some embodiments, a user selects modules from a library of modules. In some embodiments, the modules may comprise applications from GeoMx©DSP. In some embodiments, the modules may comprise applications from CosMx™ SMI. In some embodiments, the modules may comprise applications of Biomarker Discovery and Validation, Immune Profiling, Unbiased Pathway Analysis, Tissue Atlasing, Subcellular Localization Analysis, Cellular Neighborhood Analysis, Cell Atlasing, or Receptor-Ligand Interactions. In further embodiments, the modules are user-editable and/or have user-configurable parameters. Many modules are suitable. Non-limiting examples of modules are provided in Table 1.

TABLE 1
Individual methods for analysis

| Module Name | Description |
|---|---|
| Loading Data | Parse raw data files and prepare for pipeline run (Create Seurat object) |
| Arrange FOVs | Chose how to layout and arrange FOVs for display purposes |
| Quality Control | Ensure that the “raw” data adheres to defined criteria, and requirements |
| | to assess data quality |
| Normalization | Derive a normalized expression matrix from raw counts |
| SCTransform | Transform the expression matrix using the SCTranform algorithm |
| PCA | Principle Component Analysis: Get cells' projections onto leading PCs |
| Spatial Network | Create a network or graph structure of the physical distribution of cells |
| Leiden | Cluster cells using the Leiden algorithm |
| Clustering | |
| Neighborhood | Identify cells' neighbors in expression space |
| Analysis | |
| UMAP | Dimension reduction via the UMAP algorithm |
| InSituType | Cell typing using the InSituType algorithm |
| (RNA cell typing) | |
| Marker Genes | Identify marker genes associated with each cell type or cluster |
| | previously identified within a dataset. |
| Differential | Estimates and summarizes generalized linear (mixed) models for single |
| Expression | cell expression. |
| (RNA only) | |
| Spatial | identify genes which have a spatial distribution that is non- |
| Expression | uniform/autocorrelated throughout a tissue, and which may be associated |
| Analysis | with specific tissue structures, microenvironment niches, or cell types. |
| Cell Type Co- | Examine the tendency of different cell types to be located near each |
| localization | other |
| Signaling | Analysis is calculated on a per-cell basis using gene sets of pre- |
| Pathways (RNA | defined pathways. |
| only) | |
| Ligand-Receptor | Score pairs of cells and individual cells for ligand-receptor signaling |
| Analysis (RNA | and explore results. |
| only) | |
| Protein Analysis | Foundational analysis for the Protein assay |
| Subsetting | Ability to create a smaller sample data set for investigating analysis |
| | more quickly/cost effectively, for iterative purposes |
| Cell | Sub-cellular segmentation to provide subcellular structures or |
| Segmentation | regions of interest with a high degree of accuracy. |
| CELESTA | Performs cell typing by taking into account each cell's marker |
| (Protein cell | expression profile and, if necessary, spatial information. |
| typing) | |

In some embodiments, the Normalization module in Table 1 may comprise using total counts as normalization factor. In some embodiments, the Normalization module in Table 1 may comprise using Pearson Residuals. In some embodiments, the Normalization module in Table 1 may comprise generating backgraound-substracted protein data from raw mean florescence intensity (MFI) values. In some embodiments, the Quality Control module in Table 1 may be performed over Cells, Targets, or FOVs.

Spatial Network module listed in Table 1 creates a network or graph structure of the physical distribution of cells. Cells are converted to nodes in the graph, and connections between cells (e.g., nearest neighbors) are represented as edges. Network can be built in one of three ways: radius-based (all cells connected within a given radius), nearest neighbors, or Delaunay triangulation.

Quality Control module listed in Table 1 covers QC for RNA and protein assays. Application for RNA assay quality control is to flag unreliable negative probes, cells, FOVs, and target genes. The user can choose to remove those flagged negative probes, cells, target genes, and FOVs and generate a filtered dataset which is the input of the down-stream analyses. Application for protein assay quality control is to flag unreliable cells based on segmented cell area, negative probe expression, and high/low target expression. Specifically cells with outlier Grubb's test p-values (p<0.05) for segmented area are flagged. Cells with mean negative probe values below the lower threshold (default=2) or above the upper threshold (default=50) are flagged. Cells where at least a % of proteins in the bth quantile (default a=0.5, b=0.9) are considered cells with overly high target expression, and therefore flagged. Cells where fewer than c proteins are in at least the dth quantile (default c=3, d=0.5) are considered cells with low expression, and therefore flagged.

Normalization module listed in Table 1 covers both RNA and protein assays. Normalization for RNA assays is based on the concept of adjusting for library size factors to ensure that cell specific total transcript abundance and distribution of counts, which may vary some between FOVs and, more dramatically, between samples, does not influence downstream visualization and analysis of data. There are two normalization methods available: (1) Pearson's residual normalization (default) is based on the estimated mean and variance: (raw counts−mean)/sd; (2) The alternative normalization method is based on total count size factors: raw counts/total counts. Normalization for protein assays is based on the concepts of (1) background subtraction to ensure that cell specific protein expression accounts for background counts observed in IgG isotype control antibodies. (2) total intensity to reduce the effect of technical artifacts (e.g., shading/edge effects). (3) arc sin h transformation to improve visualization clarity and stabilize variance.

Principal Component analysis listed in Table 1 provides an orthogonally constrained dimensional reduction analysis of the count data across all cells in a dataset. It produces an output values (PCs) which represent axes of variation within the data which are a combined value of weighted expression within a given cell. PCs are ordered by decreasing variation explained in the data. These can be used to better understand variation within a dataset, but is most commonly used in single-cell analysis as an input for the UMAP analysis.

Uniform Manifold Approximation and Projection (UMAP) module in Table 1 UMAP provides a means to visualize high-plex complex datasets in 2-dimensional space using a non-linear approach estimating related groups of cells or features. This method is a common way of visualizing single-cell data to identify clusters of related cells which may be from the same lineage.

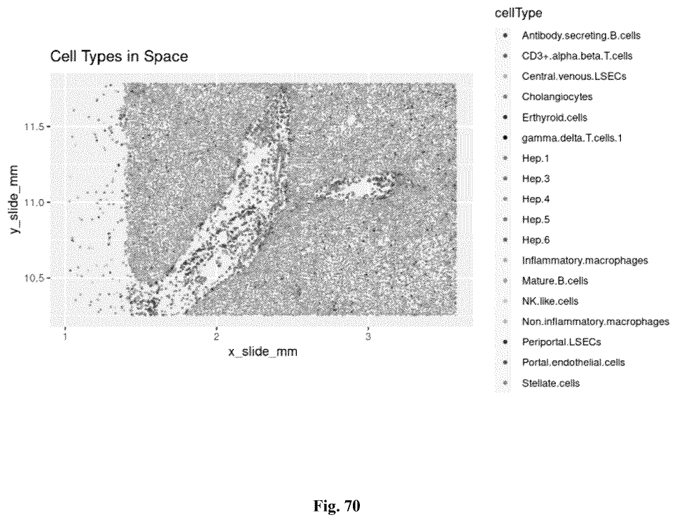

InSitu Type (RNA cell typing) module in Table 1 is a cell typing algorithm designed for statistical and computational efficiency in spatial transcriptomics data. It is based on a likelihood model that weighs the evidence from every expression value, extracting all the information available in each cell's expression profile. This likelihood model underlies a Bayes classifier for supervised cell typing, and an Expectation-Maximization algorithm for unsupervised and semi-supervised clustering. Insitutype also leverages alternative data types collected in spatial studies, such as cell images and spatial context, by using them to inform prior probabilities of cell type calls, as illustrated in FIG. 70.

CELESTA (Protein cell typing) module in Table 1 apply algorithm performs cell typing by taking into account each cell's marker expression profile and, if necessary, spatial information. Cell typing calls are guided by a signature matrix which specifies the marker(s) known to have high/low expression for each cell type. A bimodal Gaussian mixture model can then be fit to estimate the probability of each cell having “high expression” for each considered marker. When the probability is sufficiently high, a cell is considered an “anchor cell”. When the probability is not sufficiently high to make a high certainty cell type call, the algorithm also considers spatial information by taking into account the cell type calls of neighboring cells. These are considered “index cells”.

Nearest Neighbor module in Table 1 constructs a KNN (k-Nearest Neighbor) graph based on the Euclidian distance in PCA space and then constructs the SNN (Shared Nearest Network) graph with edge weights between any two cells based on the shared overlap in their local neighborhoods (Jaccard distance) and pruning of distant edges.

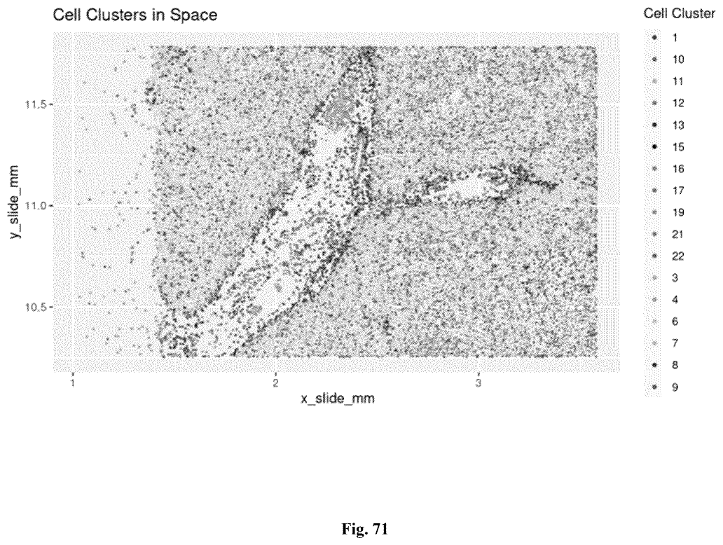

Leiden Clustering module in Table 1 is an unsupervised clustering method used to identify groups of cells which are related based on how similar they are in a graph structure. Clusters are defined by moving cells to identify groups of cells that can be aggregated without changing the overall relationship of the graph and looking for unstable nodes which serve as bridges between related communities to help define the boundaries of different clusters, as illustrated in FIG. 71.

Neighborhood Analysis module in Table 1 identifies distinct cellular neighborhood clusters based on cell type composition across tissue. This module helps define the structural composition of a tissue automatically by looking for regional differences in cell type composition. Structures can be repeated structures that are frequently found within a tissue but which are not contiguous (e.g., glomeruli in the kidney, germinal centers in the lymph node) or which are physically connected across a tissue (e.g., epithelial layer in the colon).

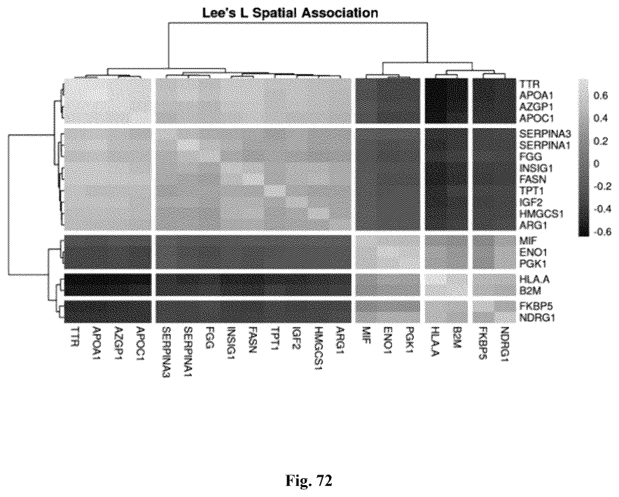

Spatial Expression Analysis module in Table 1 is used to identify genes which have a spatial distribution that is non-uniform/autocorrelated throughout a tissue, and which may be associated with specific tissue structures, microenvironment niches, or cell types. Use this module to look for genes which carry spatially-relevant information. The module also measures associated spatial expression between genes which can be used to group genes into different spatial expression patterns. The two statistics calculated related to spatial expression patterns are Moran's I and Lee's L. FIG. 72 illustrates spatial expression patterns based on Lee's L.

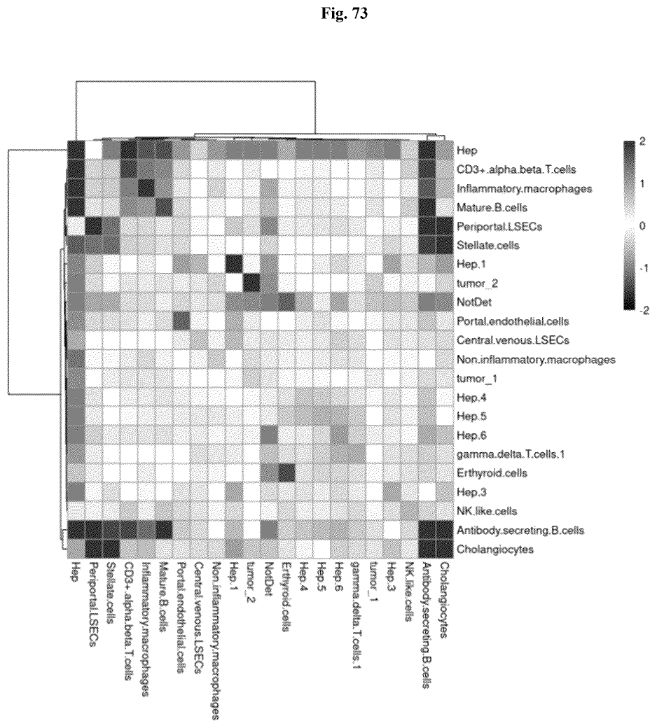

Cell Type co-localization module in Table 1 examines the tendency of different cell types to be located near each other. Each pair of cell types defined from supervised or unsupervised clustering is tested using Ripley's K-function (a function of the distance between the different cell types) for whether the cells' spatial distribution differs from a theoretical Poisson point process where a cell's location is not dependent on another cell's location. The results are summarized in a heatmap indicating which cell types tend to cluster together or isolate from each other as illustrated in FIG. 73. In addition, a more granular view is shown when plotting the pair correlation function for a given cell type pairing as a function of the radius which can reveal specific distances at which the cells of each type are co-localized.

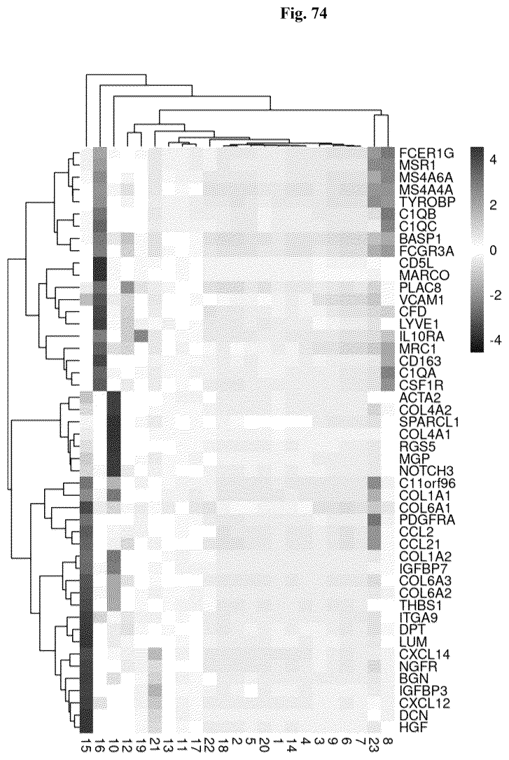

Marker Genes module in Table 1 will identify marker genes associated with each cell type or cluster previously identified within a dataset. This module looks for genes which are expressed above background consistently, but also most specifically restricted to each cell type or cluster within the dataset. The module acts on each gene independently. This module may also be used to look for marker genes in neighborhoods that have been identified, but these genes will be related to the overall cellular composition of those neighborhoods, as illustrated in FIG. 74.

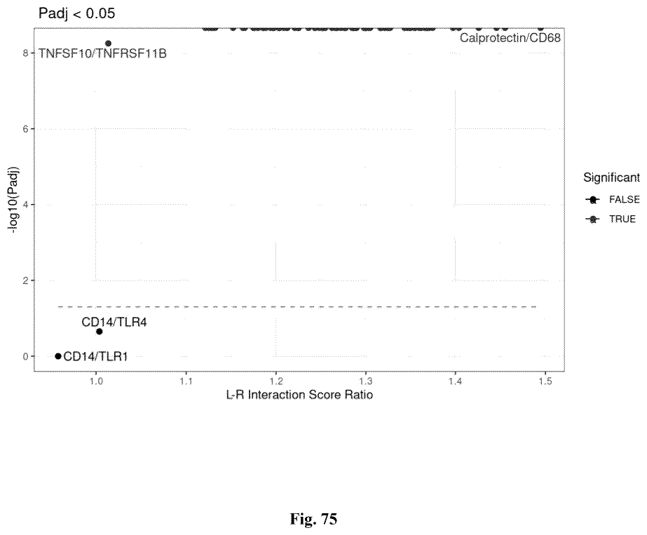

Ligand-Receptor Analysis (RNA only) module in Table 1 scores pairs of cells and individual cells for ligand-receptor signaling and explore results. Ligand-receptor target expression in adjacent cells is used to calculate a co-expression score. A test is then performed to determine if the overall average of the scores for each ligated-receptor pair is enriched by the spatial arrangement of cells, as illustrated in FIG. 75.

Signaling Pathways (RNA only) module in Table 1 is calculated on a per-cell basis using gene sets of pre-defined pathways. The method calculates relative enrichment of a pathway of the genes within each pathway. Gene sets which do not have sufficient coverage are excluded from analysis.

Differential Expression (RNA only) module in Table 1 estimates and summarizes generalized linear (mixed) models for single cell expression. The expression of neighboring cells are controlled on a tissue by including ‘neighboring cell expression’ of the analyzed gene as a fixed-effect control variable in the DE model. Controlling for expression in neighboring cells is motivated by the observation that cells in close proximity on a tissue are not independent, and comparisons of DE between groups may be affected by cell-segmentation and cell-type uncertainty. Single cell DE analyses may test whether groups are DE within a specific cell-type. In practice, imperfect cell-segmentation can result in ‘contamination’ or ‘bleed-over’ of transcripts from neighboring cells, which can also increase uncertainty in downstream cell-typing analyses. For these reasons, including the expression of the analyzed gene in neighboring cells can be a useful control variable.

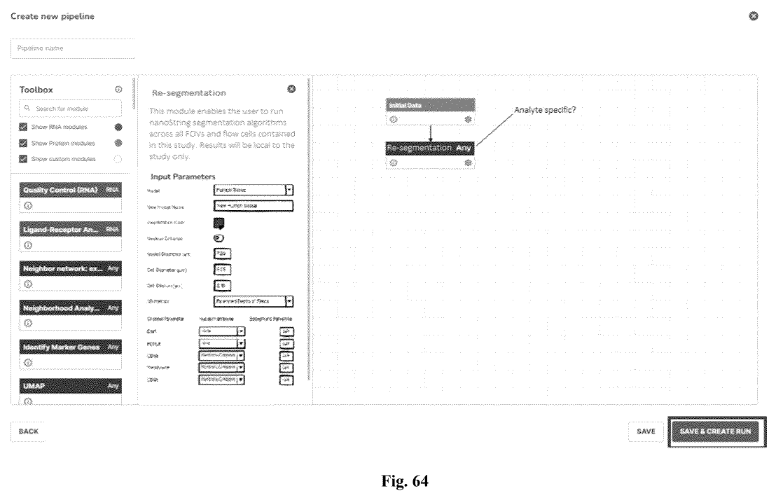

FIG. 64 shows a non-limiting example of user interface of the cell segmentation module on pipeline orchestrator. One or more flow cells can be selected for study creation and displayed on the cell segmentation module. The pipeline orchestrator handles the loading of the morphology images and sends them to the cell segmentation module. In some embodiments, the cell segmentation module comprises one or more deep learning models. In some embodiments, one or more deep learning models are trained using images of cells or tissues. In some embodiments, images of cells or tissues comprise microscope-derived images, morphology images, or fluorescence images. In some embodiments, a deep learning model is trained suitable to segment a particular tissue type.

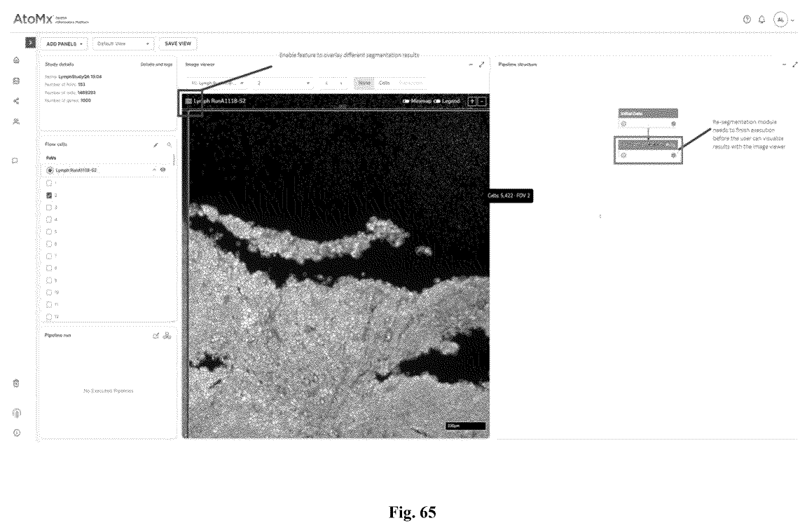

An inference engine will be responsible for loading the model and segment cells from the input images of cells and tissues based on the model predictions in cell segmentation module. Then a model selection function will be responsible for selecting the best model for the given input image. Users can modify various cell segmentation parameters such as cell diameter, dilation parameter, cell probability and gradient flow threshold over the parameter selection interface. The pipeline orchestrator saves these sets of parameters to current study. The pipeline can support multiple segmentation results. The pipeline orchestrator will provide methods of communication between the cell segmentation module and image viewer to display cell segmentation results overlay. FIG. 65 shows the integration of cell segmentation output into the image viewer. The pipeline orchestrator will differentiate between transitory state to permanent state of cell segmentation results while user interacts with the module and changing the parameters. The pipeline is pre-loaded with the default parameters and each flow cell segmentation results can be re-instated to its initial default state. In some embodiments, image viewer is enabled to overlay different segmentation results. In completing one cell re-segmentation event, the pipeline orchestrator will trigger transcript to cell reassignment logic, which will trigger the rest of the data analysis in the pipeline. In some embodiments, re-segmentation module may finish execution before the user can visualize results with the image viewer.

In some embodiments, wherein one or more modules available in the pipeline orchestrator tool comprises training a machine learning model and/or applying a machine learning model. In some embodiments, the machine learning model may comprise dimension reduction through a nonlinear dimensionality reduction algorithm. In some embodiments, the nonlinear dimensionality reduction algorithm may comprise Sammon's mapping, Principal curves and manifolds, Laplacian eigenmaps, Isomap, Locally-linear embedding, Local tangent space alignment, Maximum variance unfolding, Gaussian process latent variable models, t-distributed stochastic neighbor embedding, Relation perspective map, Contagion maps, Curvilinear component analysis, Curvilinear distance analysis, Diffeomorphic dimensionality reduction, Manifold alignment, Diffusion maps, Local multidimensional scaling, Nonlinear PCA, Data-driven high-dimensional scaling, Manifold sculpting, RankVisu, Topologically constrained isometric embedding, Uniform manifold approximation and projection (UMAP). In some embodiments, the UMAP module in Table 1 may apply a feed-forward neural network (e.g., an autoencoder) on a subset of the data to project the manifold clustering onto the entire dataset. In some embodiments, the feed-forward neural network may be trained to approximate the identity function (i.e., trained to map from a vector of values to the same vector). In some embodiments, the feed-forward neural network may be used for dimensionality reduction purpose, wherein one of the hidden layers in the networks may be limited to contain only a small number of network units.

In some embodiments, the machine learning may comprise approaches in combining of method for estimating probability distribution. In some embodiments, the Cell Typing module in Table 1 may apply an approach of maximum likelihood estimation to clustering based on known gene expression profiles. In some embodiments, the Cell Typing module may be a standalone application.

In some embodiments, pipeline orchestrator tool may further comprise a data ingestion process. In some embodiments, the data ingestion process may produce a set of the output files comprising summary information from the image data from laboratory instruments. In some embodiments, user may select groups of data files organized by flowcells within pipeline orchestrator tool. In some other embodiments, the data ingestion process may produce a set of tileDB arrays connected to a Seurat object. In some further embodiments, user may perform analysis for the arrays. In some further embodiments, the analysis results within the orchestrator may be written back into the Seurat/tileDB object. In some embodiments, Raw files are exported. In some embodiments, Raw files are exported once per study.

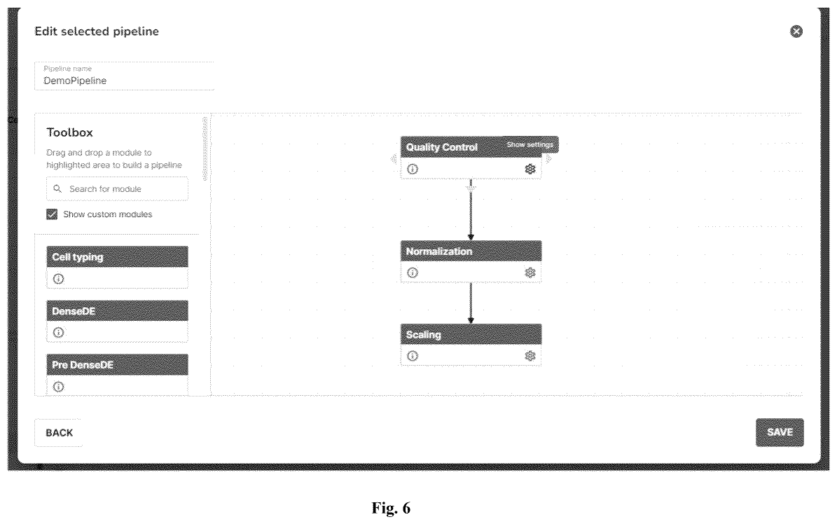

FIG. 6 shows a non-limiting example of a GUI for a spatial biology informatics integration portal; in this case, a GUI including a pipeline editing environment for a pipeline orchestrator allowing a user to edit pipelines and/or module run parameters. Referring to FIG. 6, the module toolbox includes modules for cell typing, DenseDE, Pre DenseDE, and the like. Continuing to refer to FIG. 6, a user has named the pipeline and has placed three modules, quality control, normalization, and scaling, linked serially into a pipeline. Each module includes an icon allowing the user to access settings.

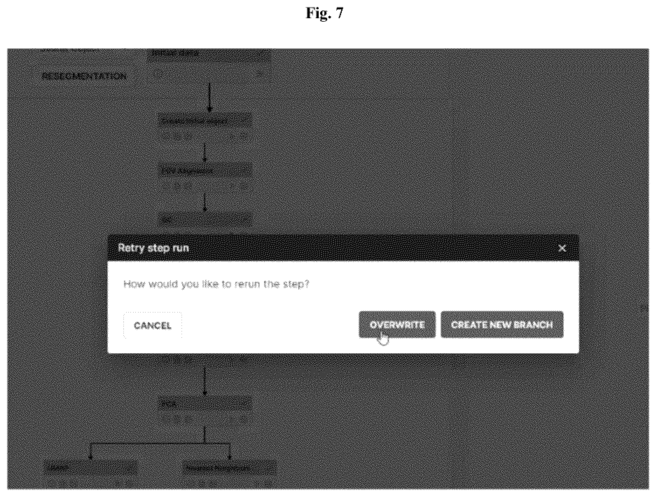

FIG. 7 shows a non-limiting example of a GUI for a spatial biology informatics integration portal including a pipeline branching feature for a pipeline orchestrator allowing a user to perform iterative analysis by modifying parameters and re-running a step or creating a new branch of the same pipeline. Modules are optionally configured to run in series, in parallel, and/or in branching pipelines.

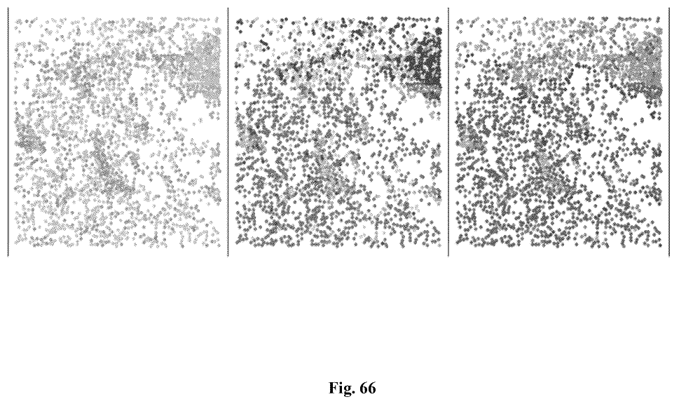

FIG. 66 shows a non-limiting example of a display from Cell Typing module. CosMx™ is a spatial molecular imager for sub-cellular resolution of mRNA transcripts and protein targets. A subset of the CosMx™ NSCLC showcase dataset is analyzed based on semi-supervised clustering. Pipeline orchestrator allows a user to perform data from mRNA transcripts and protein targets and integrate the data and analysis. Cell typing is displayed via coloring based on the frequency of cell types in the decision.

### Machine Learning

In some embodiments, the platforms, systems, media, and methods disclosed herein include one or more modules available in the pipeline orchestrator tool comprises training a machine learning model and/or applying a machine learning model. In some embodiments, the machine learning model may comprise an unsupervised machine learning model, a supervised machine learning model, semi-supervised machine learning model, or a self-supervised machine learning. In some embodiments, the supervised machine learning model may comprise, for example, a Random Forest, a support vector machine (SVM), a neural network, or a deep learning algorithm.

In some embodiments, the platforms, systems, media, and methods disclosed herein include a machine learning model utilizes one or more neural networks. In some embodiments, a neural network is a type of computational system that can learn the relationships between an input dataset and a target dataset. A neural network may be a software representation of a human neural system (e.g., cognitive system), intended to capture “learning” and “generalization” abilities as used by a human. In some embodiments, the machine learning algorithm comprises a neural network comprising a CNN. Non-limiting examples of structural components of machine learning algorithms described herein include: CNNs, recurrent neural networks, dilated CNNs, fully-connected neural networks, deep generative models, and Boltzmann machines.

In some embodiments, a neural network comprises a series of layers termed “neurons.” In some embodiments, a neural network comprises an input layer, to which data is presented; one or more internal, and/or “hidden”, layers; and an output layer. A neuron may be connected to neurons in other layers via connections that have weights, which are parameters that control the strength of the connection. The number of neurons in each layer may be related to the complexity of the problem to be solved. The minimum number of neurons required in a layer may be determined by the problem complexity, and the maximum number may be limited by the ability of the neural network to generalize. The input neurons may receive data being presented and then transmit that data to the first hidden layer through connections' weights, which are modified during training. The first hidden layer may process the data and transmit its result to the next layer through a second set of weighted connections. Each subsequent layer may “pool” the results from the previous layers into more complex relationships. In addition, whereas conventional software programs require writing specific instructions to perform a function, neural networks are programmed by training them with a known sample set and allowing them to modify themselves during (and after) training so as to provide a desired output such as an output value. After training, when a neural network is presented with new input data, it is configured to generalize what was “learned” during training and apply what was learned from training to the new previously unseen input data in order to generate an output associated with that input.

In some embodiments, the neural network comprises artificial neural networks (ANNs). ANNs may be machine learning algorithms that may be trained to map an input dataset to an output dataset, where the ANN comprises an interconnected group of nodes organized into multiple layers of nodes. For example, the ANN architecture may comprise at least an input layer, one or more hidden layers, and an output layer. The ANN may comprise any total number of layers, and any number of hidden layers, where the hidden layers function as trainable feature extractors that allow mapping of a set of input data to an output value or set of output values. As used herein, a deep learning algorithm (such as a deep neural network (DNN)) is an ANN comprising a plurality of hidden layers, e.g., two or more hidden layers. Each layer of the neural network may comprise a number of nodes (or “neurons”). A node receives input that comes either directly from the input data or the output of nodes in previous layers, and performs a specific operation, e.g., a summation operation. A connection from an input to a node is associated with a weight (or weighting factor).

The node may sum up the products of all pairs of inputs and their associated weights. The weighted sum may be offset with a bias. The output of a node or neuron may be gated using a threshold or activation function. The activation function may be a linear or non-linear function. The activation function may be, for example, a rectified linear unit (ReLU) activation function, a Leaky ReLU activation function, or other function such as a saturating hyperbolic tangent, identity, binary step, logistic, arctan, softsign, parametric rectified linear unit, exponential linear unit, softplus, bent identity, softexponential, sinusoid, sinc, Gaussian, or sigmoid function, or any combination thereof.

The weighting factors, bias values, and threshold values, or other computational parameters of the neural network, may be “taught” or “learned” in a training phase using one or more sets of training data. For example, the parameters may be trained using the input data from a training dataset and a gradient descent or backward propagation method so that the output value(s) that the ANN computes are consistent with the examples included in the training dataset.

The number of nodes used in the input layer of the ANN or DNN may be at least about 10, 50, 100, 200, 300, 400, 500, 600, 700, 800, 900, 1000, 2000, 3000, 4000, 5000, 6000, 7000, 8000, 9000, 10,000, 20,000, 30,000, 40,000, 50,000, 60,000, 70,000, 80,000, 90,000, 100,000, or greater.

In other instances, the number of nodes used in the input layer may be at most about 100,000, 90,000, 80,000, 70,000, 60,000, 50,000, 40,000, 30,000, 20,000, 10,000, 9000, 8000, 7000, 6000, 5000, 4000, 3000, 2000, 1000, 900, 800, 700, 600, 500, 400, 300, 200, 100, 50, 10, or less. In some instances, the total number of layers used in the ANN or DNN (including input and output layers) may be at least about 3, 4, 5, 10, 15, 20, or greater. In other instances, the total number of layers may be at most about 20, 15, 10, 5, 4, 3, or less.

In some instances, the total number of learnable or trainable parameters, e.g., weighting factors, biases, or threshold values, used in the ANN or DNN may be at least about 10, 50, 100, 200, 300, 400, 500, 600, 700, 800, 900, 1000, 2000, 3000, 4000, 5000, 6000, 7000, 8000, 9000, 10,000, 20,000, 30,000, 40,000, 50,000, 60,000, 70,000, 80,000, 90,000, 100,000, or greater. In other instances, the number of learnable parameters may be at most about 100,000, 90,000, 80,000, 70,000, 60,000, 50,000, 40,000, 30,000, 20,000, 10,000, 9000, 8000, 7000, 6000, 5000, 4000, 3000, 2000, 1000, 900, 800, 700, 600, 500, 400, 300, 200, 100, 50, 10, or less.

In some embodiments, the platforms, systems, media, and methods disclosed herein include a machine learning model comprises a neural network such as a deep CNN. In some embodiments in which a CNN is used, the network is constructed with any number of convolutional layers, dilated layers or fully-connected layers. In some embodiments, the number of convolutional layers is between 1-10 and the dilated layers between 0-10. The total number of convolutional layers (including input and output layers) may be at least about 1, 2, 3, 4, 5, 10, 15, 20, or greater, and the total number of dilated layers may be at least about 1, 2, 3, 4, 5, 10, 15, 20, or greater. The total number of convolutional layers may be at most about 20, 15, 10, 5, 4, 3, or less, and the total number of dilated layers may be at most about 20, 15, 10, 5, 4, 3, or less. In some embodiments, the number of convolutional layers is between 1-10 and the fully-connected layers between 0-10. The total number of convolutional layers (including input and output layers) may be at least about 1, 2, 3, 4, 5, 10, 15, 20, or greater, and the total number of fully-connected layers may be at least about 1, 2, 3, 4, 5, 10, 15, 20, or greater. The total number of convolutional layers may be at most about 20, 15, 10, 5, 4, 3, 2, 1, or less, and the total number of fully-connected layers may be at most about 20, 15, 10, 5, 4, 3, 2, 1, or less.

In some embodiments, a machine learning algorithm comprises a neural network comprising a convolutional neural network (CNN), a recurrent neural network (RNN), dilated CNN, fully-connected neural networks, deep generative models and/or deep restricted Boltzmann machines.

In some embodiments, a machine learning model comprises one or more CNNs. The CNN may be deep and feedforward ANNs. The CNN may be applicable to analyzing visual imagery. The CNN may comprise an input, an output layer, and multiple hidden layers. The hidden layers of a CNN may comprise convolutional layers, pooling layers, fully-connected layers and normalization layers. The layers may be organized in 3 dimensions: width, height, and depth.

The convolutional layers may apply a convolution operation to the input and pass results of the convolution operation to the next layer. For processing images, the convolution operation may reduce the number of free parameters, allowing the network to be deeper with fewer parameters. In neural networks, each neuron may receive input from some number of locations in the previous layer. In a convolutional layer, neurons may receive input from only a restricted subarea of the previous layer. The convolutional layer's parameters may comprise a set of learnable filters (or kernels). The learnable filters may have a small receptive field and extend through the full depth of the input volume. During the forward pass, each filter may be convolved across the width and height of the input volume, compute the dot product between the entries of the filter and the input, and produce a two-dimensional activation map of that filter. As a result, the network may learn filters that activate when it detects some specific type of feature at some spatial position in the input.

In some embodiments, a machine learning model comprises an RNN. RNNs are neural networks with cyclical connections that can encode and process sequential data. An RNN can include an input layer that is configured to receive a sequence of inputs. An RNN may additionally include one or more hidden recurrent layers that maintain a state. At each step, each hidden recurrent layer can compute an output and a next state for the layer. The next sate may depend on the previous state and the current input. The state may be maintained across steps and may capture dependencies in the input sequence.

An RNN can be a long short-term memory (LSTM) network. An LSTM network may be made of LSTM units. An LSTM unit may include of a cell, an input gate, an output gate, and a forget gate. The cell may be responsible for keeping track of the dependencies between the elements in the input sequence. The input gate can control the extent to which a new value flows into the cell, the forget gate can control the extent to which a value remains in the cell, and the output gate can control the extent to which the value in the cell is used to compute the output activation of the LSTM unit.

Alternatively, an attention mechanism (e.g., a transformer). Attention mechanisms may focus on, or “attend to,” certain input regions while ignoring others. This may increase model performance because certain input regions may be less relevant. At each step, an attention unit can compute a dot product of a context vector and the input at the step, among other operations. The output of the attention unit may define where the most relevant information in the input sequence is located.

In some embodiments, the pooling layers comprise global pooling layers. The global pooling layers may combine the outputs of neuron clusters at one layer into a single neuron in the next layer. For example, max pooling layers may use the maximum value from each of a cluster of neurons in the prior layer; and average pooling layers may use the average value from each of a cluster of neurons at the prior layer.

In some embodiments, the fully-connected layers connect every neuron in one layer to every neuron in another layer. In neural networks, each neuron may receive input from some number locations in the previous layer. In a fully-connected layer, each neuron may receive input from every element of the previous layer.

In some embodiments, the normalization layer is a batch normalization layer. The batch normalization layer may improve the performance and stability of neural networks. The batch normalization layer may provide any layer in a neural network with inputs that are zero mean/unit variance. The advantages of using batch normalization layer may include faster trained networks, higher learning rates, easier to initialize weights, more activation functions viable, and simpler process of creating deep networks.

The trained algorithm may be configured to accept a plurality of input variables and to produce one or more output values based on the plurality of input variables. The trained algorithm may comprise a classifier, such that each of the one or more output values comprises one of a fixed number of possible values (e.g., a linear classifier, a logistic regression classifier, etc.) indicating a classification of the biological sample and/or the subject by the classifier. The trained algorithm may comprise a binary classifier, such that each of the one or more output values comprises one of two values (e.g., {0, 1}, {positive, negative}, or {high-risk, low-risk}) indicating a classification of the biological sample and/or subject by the classifier. The trained algorithm may be another type of classifier, such that each of the one or more output values comprises one of more than two values (e.g., {0, 1, 2}, {positive, negative, or indeterminate}, or {high-risk, intermediate-risk, or low-risk}) indicating a classification of the biological sample and/or subject by the classifier. The output values may comprise descriptive labels, numerical values, or a combination thereof. Some of the output values may comprise descriptive labels. Some of the output values may comprise numerical values, such as binary, integer, or continuous values. Such binary output values may comprise, for example, {0, 1}, {positive, negative}, or {high-risk, low-risk}. Such integer output values may comprise, for example, {0, 1, 2}. Such continuous output values may comprise, for example, a probability value of at least 0 and no more than 1. Such continuous output values may comprise, for example, an un-normalized probability value of at least 0. Such continuous output values may indicate a prognosis of the cancer-related category of the subject. Some numerical values may be mapped to descriptive labels, for example, by mapping 1 to “positive” and 0 to “negative.”.

The classification of samples may assign an output value of “indeterminate” or 2 if the sample is not classified as “positive”, “negative”, 1, or 0. In this case, a set of two cutoff values is used to classify samples into one of the three possible output values. Examples of sets of cutoff values may include {1%, 99%}, {2%, 98%}, {5%, 95%}, {10%, 90%}, {15%, 85%}, {20%, 80%}, {25%, 75%}{30%, 70%}, {35%, 65%}, {40%, 60%}, and {45%, 55%}. Similarly, sets of n cutoff values may be used to classify samples into one of n+1 possible output values, where n is any positive integer.

### Visualization

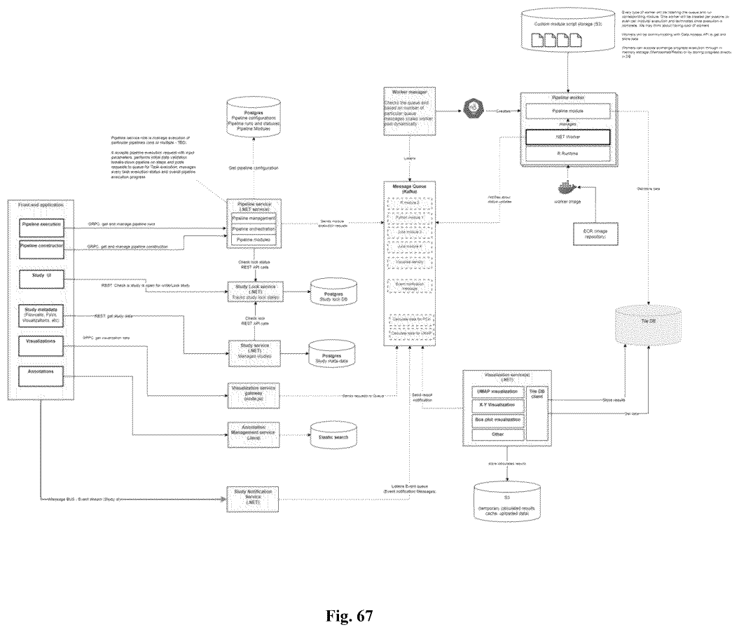

FIG. 67 shows a non-limiting example of overview of the application architecture and services of for development of graphic processing. The core application services are listed at the center and visualization services are diagrammed in the bottom right. Front-end application modules are listed on the left side. Pipeline service (Pipeline Management Service) takes inputs from Pineline execution and Pipeline constructor from front-end application, manages pipelines and pipeline run execution process. Study Lock Service follows input from Study UI and is responsible for controlling study lock status and managing active study locks. Study service is responsible for managing studies and study creation process. It takes input of study metadata in forms of flowcells, FoVs, visualizations, as well as others. Visualization Service Gateway is the main gateway for visualization data preparation taking data from the front-end module. Visualization Service Worker prepares data for visualizations and communicates with Visualization Service Gateway through message queue, which is illustrated in the middle right of FIG. 67. Annotation Management service is responsible for annotation management and annotation search. Study Notification Service provides access to notification message stream related to a specific study. As shown in FIG. 67 on top right, Worker Manager is responsible for managing the life-cycle of pipeline processing workers. Pipeline Worker is responsible for running pipeline steps or custom modules.

In some embodiments, the platforms, systems, media, and methods disclosed herein include features and functionality to generate a data summary, result, and/or visualization. In some embodiments, a summary, result, and/or visualization is generated for data received from laboratory equipment. In some embodiments, a summary, result, and/or visualization is generated for data transformed via one or more modules or pipelines. In some embodiments, visualization display may comprise graphs, plots, and overlay points representing transcripts or cell annotations onto tissue images. In some embodiments, visualization may query that Seurat/tileDB object to retrieve the relevant data from the object for display. In some further embodiments, the relevant data may comprise transcript locations and/or cell annotations. In some embodiments, visualization may be displayed as transcript locations as points, cell annotations (e.g., cell type) as points, boxplots, violin plots, dot plots.

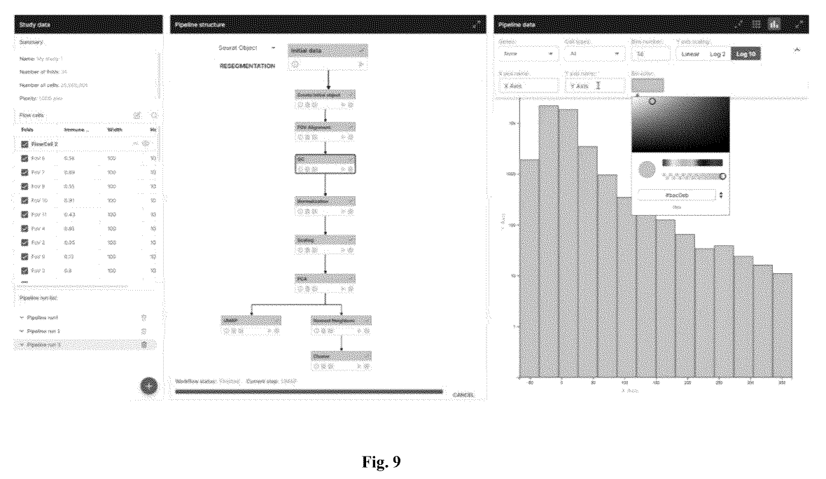

FIG. 9 shows a non-limiting example of a GUI for a spatial biology informatics integration portal including an environment for viewing an analysis pipeline for a study as well as visualizing results of modules of the pipeline and/or the pipeline. Referring to FIG. 9, the pipeline run list includes three pipeline runs. The current pipeline includes an initial data module, a create linked object module, a FoV alignment module, a QC module, a normalization module, a scaling module, a PCA module, which branches to a UMAP module and a nearest neighbors module followed by a cluster module. Continuing to refer to FIG. 9, the QC module is selected and the pipeline data section includes a data summary/visualization (X-Y plot with Log 10 Y-axis scaling).

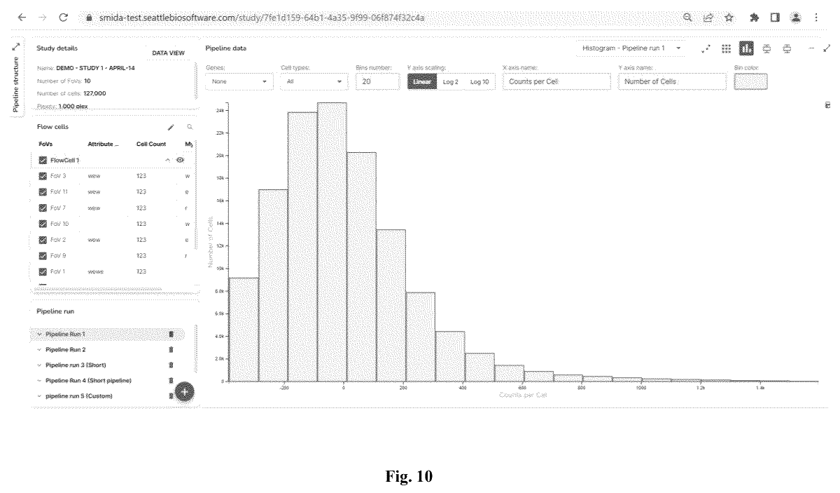

FIG. 10 shows a non-limiting example of a GUI for a spatial biology informatics integration portal including customizable layout options, such as, variable sizing of different windowpanes (such as the study details pane, the pipeline structure pane, and the pipeline data pane).

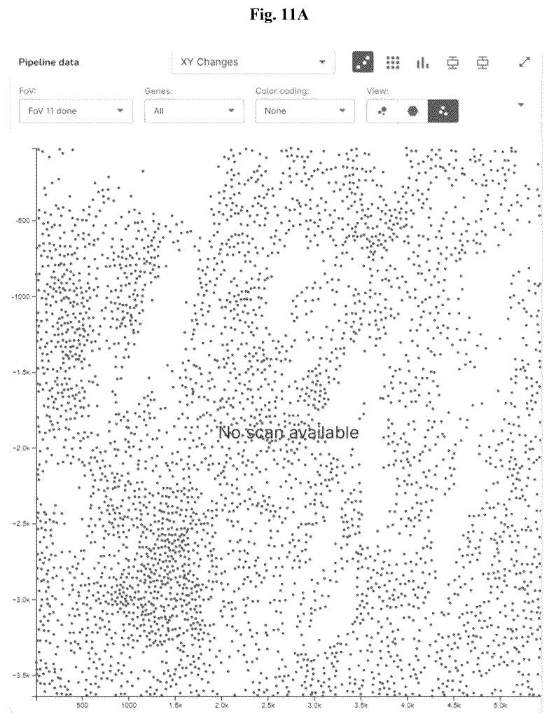

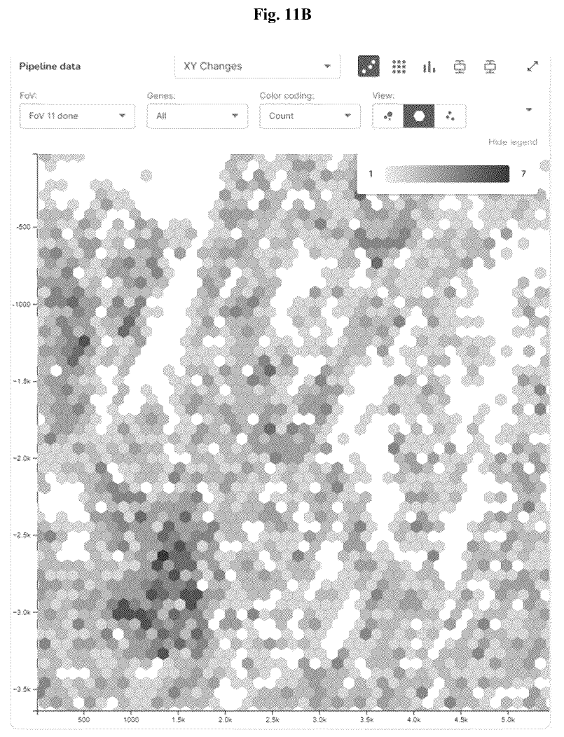

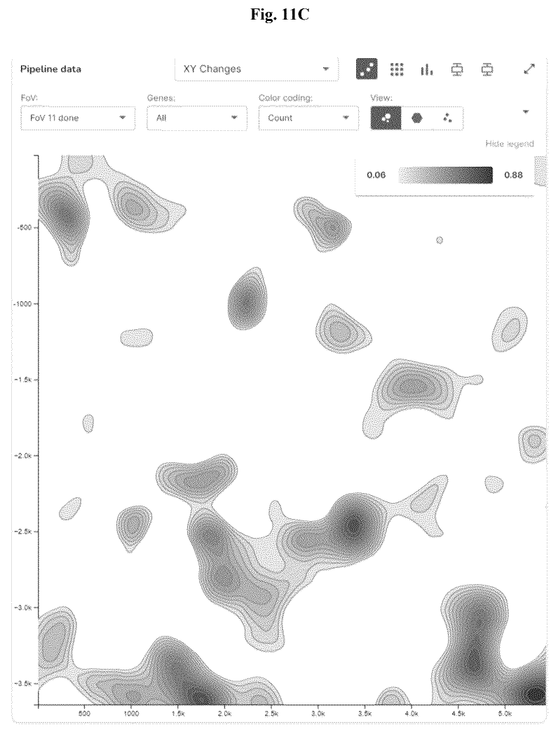

FIGS. 11A-11C show a non-limiting example of a GUI for a spatial biology informatics integration portal including an environment to graphically represent pipeline data as X-Y scatterplots of, for example, cell or transcript coordinates for a particular FoV associated with a study. Configurable options include selection of FoV(s), selection of gene(s), color coding, type of visualization, and view.

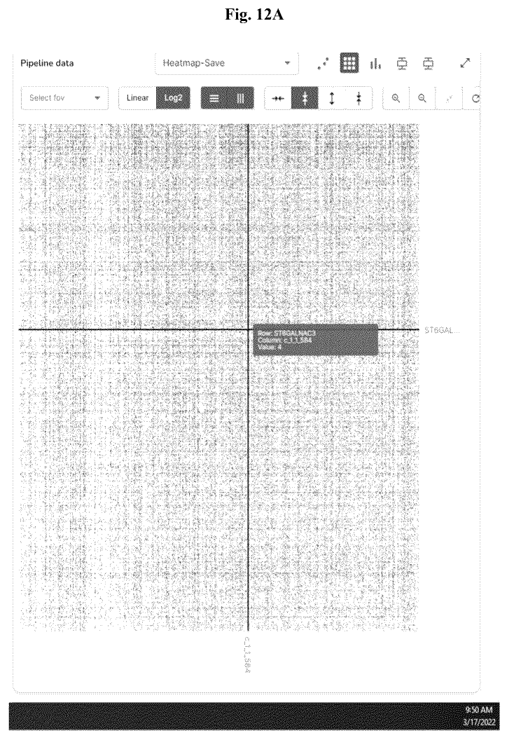

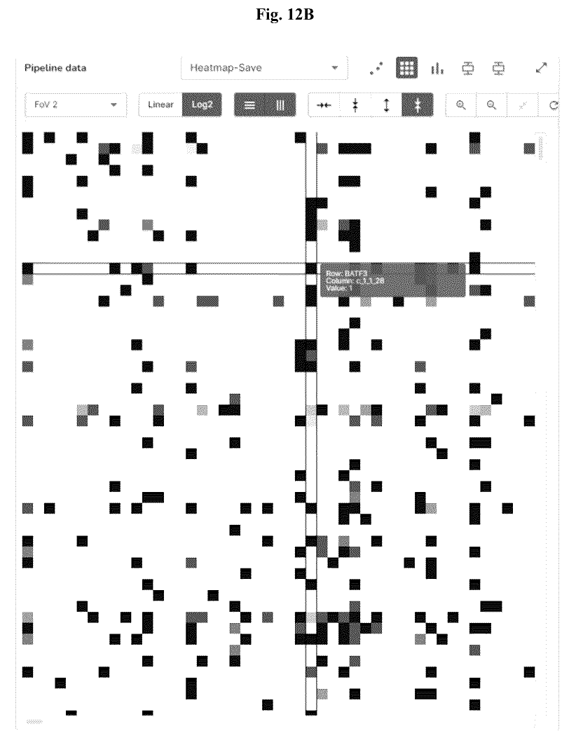

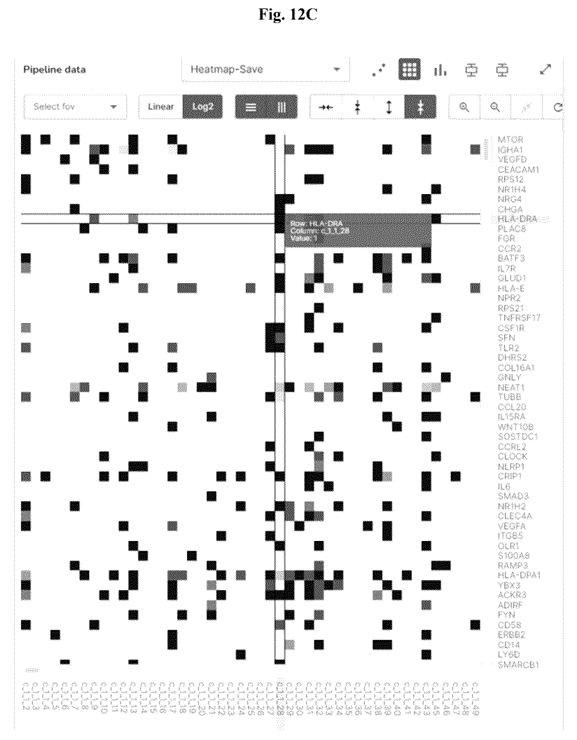

FIGS. 12A-12C show a non-limiting example of a GUI for a spatial biology informatics integration portal including an environment to graphically represent pipeline data as heatmaps for a particular FoV associated with a study. Configurable options include selection of FoV(s), type of visualization, and scaling.

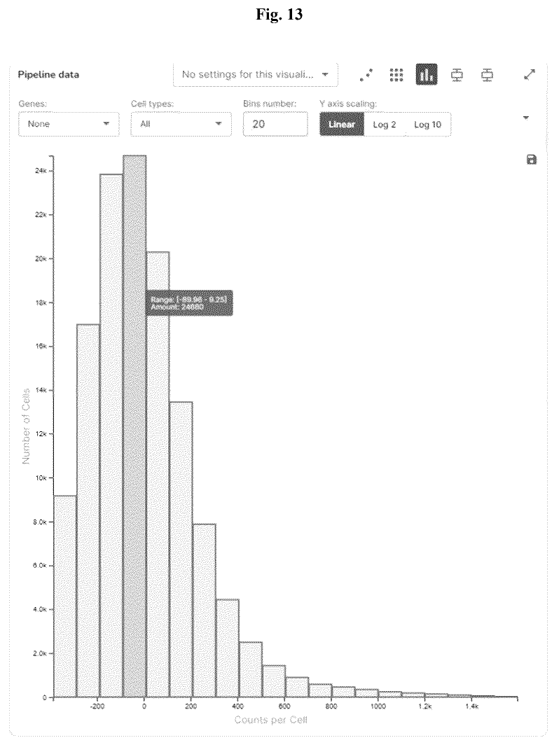

FIG. 13 shows a non-limiting example of a GUI for a spatial biology informatics integration portal including an environment to graphically represent pipeline data as histograms. Configurable options include, selection of gene(s), selection of cell type(s), type of visualization, and Y-axis scaling.

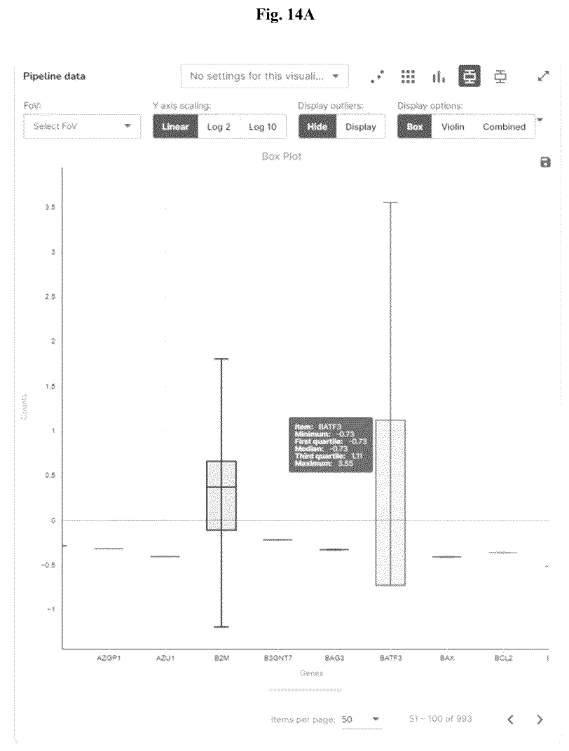

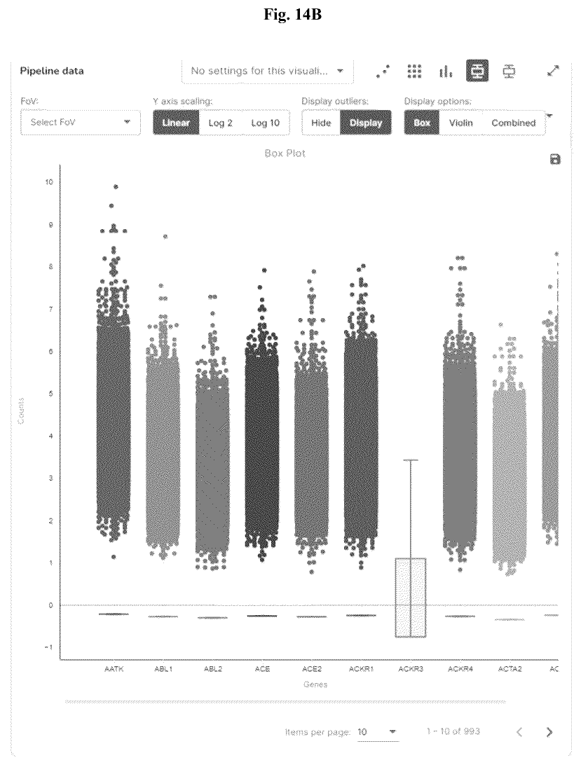

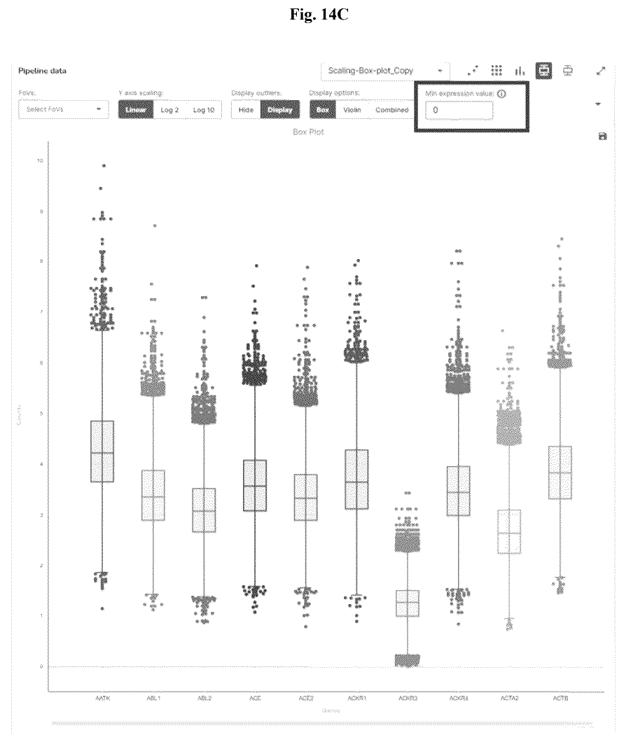

FIGS. 14A-14C show a non-limiting example of a GUI for a spatial biology informatics integration portal including an environment to graphically represent pipeline data as boxplots for a particular FoV associated with a study. Configurable options include selection of FoV(s), Y-axis scaling, display of outliers, and type of visualization.

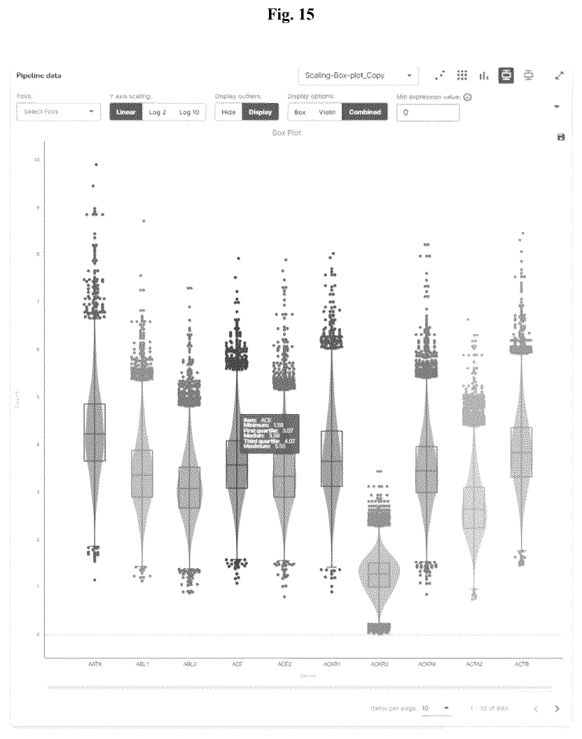

FIG. 15 shows a non-limiting example of a GUI for a spatial biology informatics integration portal including an environment to graphically represent pipeline data as violin plots. Configurable options include selection of FoV(s), Y-axis scaling, display of outliers, type of visualization, and minimum expression value.

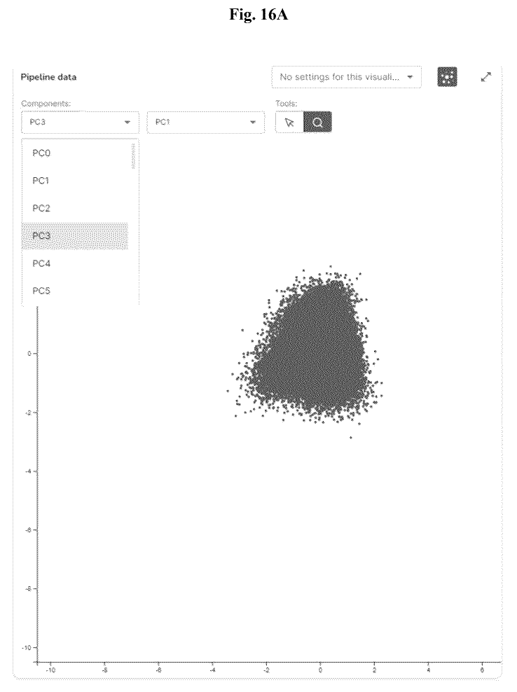

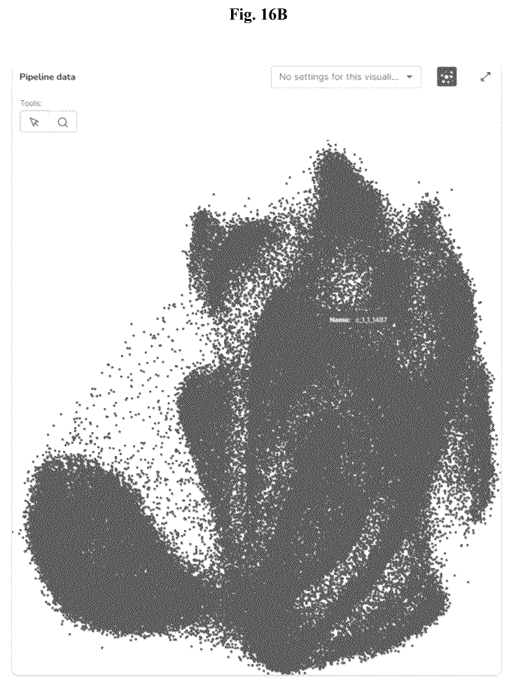

FIGS. 16A and 16B show a non-limiting example of a GUI for a spatial biology informatics integration portal including an environment to perform dimension reduction of pipeline data including by, for example, Principal Component Analysis (PCA) and Uniform Manifold Approximation and Projection (UMAP). Configurable options include selection of components.

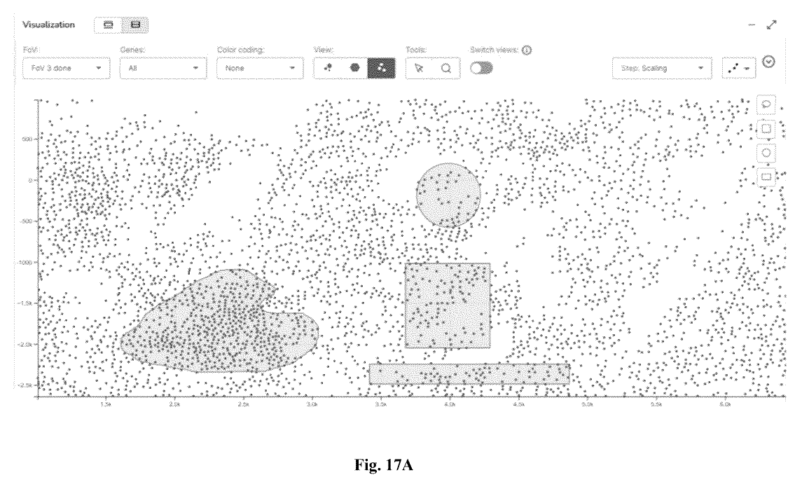

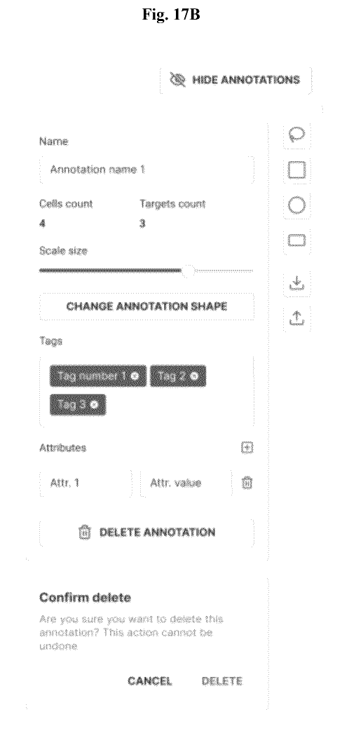

FIGS. 17A and 17B shows a non-limiting example of a GUI for a spatial biology informatics integration portal including an environment allowing a user to annotate pipeline data by using drawing tools to identify regions of images and/or graphical plots. Configurable options include selection of FoV(s), selection of gene(s), color coding, and view. Referring to FIG. 17A, the user optionally draws geometric shapes to identify data to annotate or draws a freehand shape to identify data to annotate. Now referring to FIG. 17B, the user optionally names an annotation, scales the size of the region identified, changes the shape used to identify the region, adds information tags to the annotation, assigns attributes to the annotation, and/or deletes the annotation.

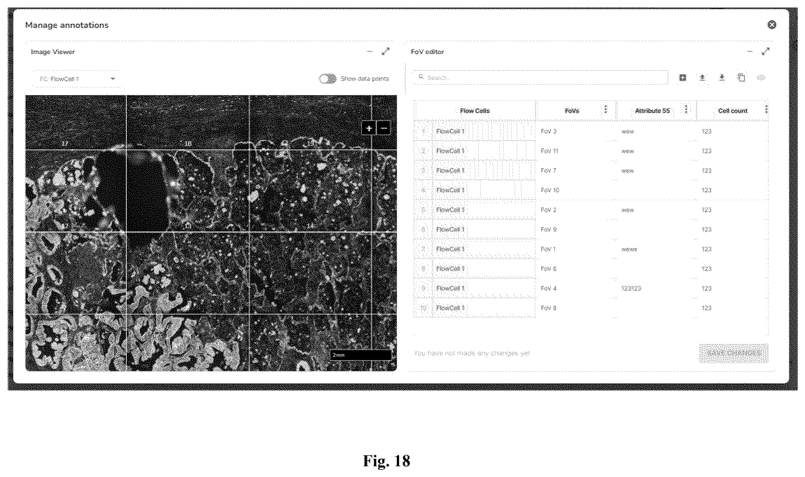

FIG. 18 shows a non-limiting example of a GUI for a spatial biology informatics integration portal including a test annotation service for interactive annotations linked to a flow cell image and table. Referring to FIG. 18, the interface allows a user to manage annotations and comprises an image viewer and a FoV editor.

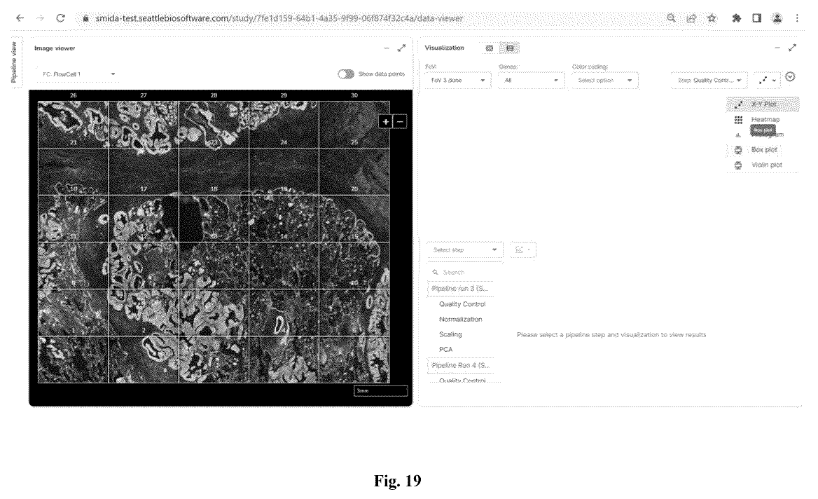

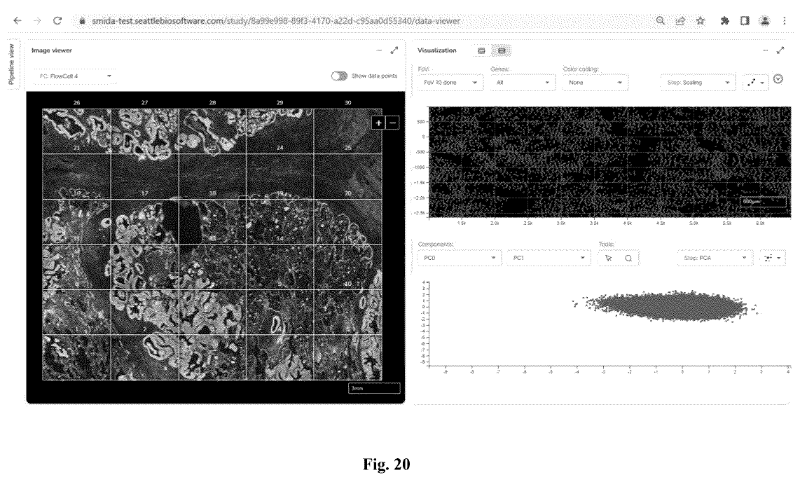

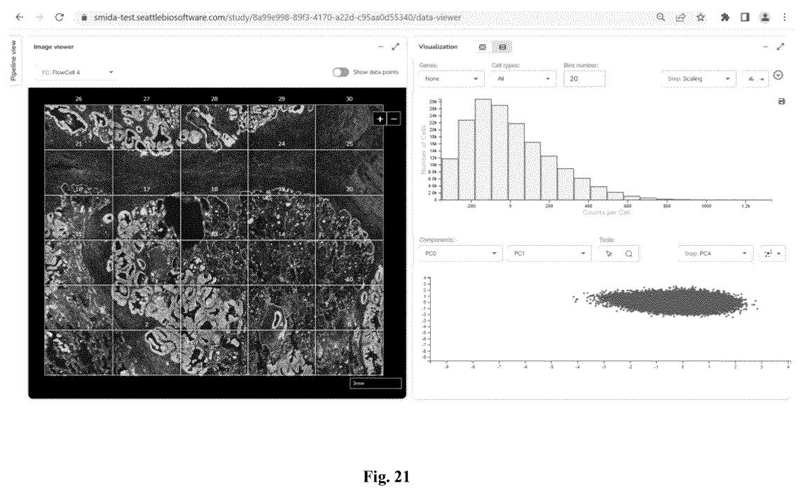

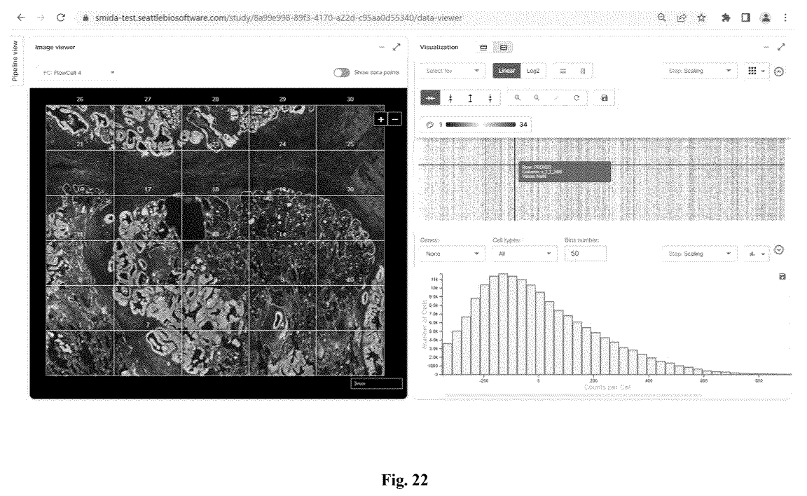

FIGS. 19-22 show a non-limiting example of a GUI for a spatial biology informatics integration portal including an environment for interactive pipeline data viewing allowing a user to select a flow cell image and visualize relevant data using any running pipeline module.

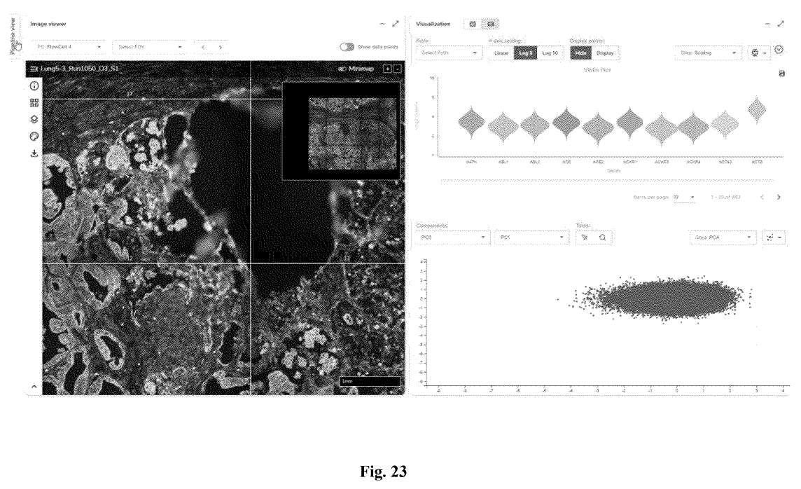

FIG. 23 shows a non-limiting example of a GUI for a spatial biology informatics integration portal including an environment for data visualization with image viewer integration allowing a user to control the image viewer display and see updates to relevant visualized data.

### Data Export Module Outputs

In some embodiments, dataset exported from the AtoMx™ Spatial Informatics Platform (SIP) is further analyzed. In some embodiments, data from SIP is stored as both a standalone Seurat object and an equivalent TileDB array. In some embodiments, exported data comprises transcript counts/location, field of view images, annotation metadata, or user-initiated data transformations performed in AtoMx SIP prior to export.

The export module can be used at any point in a pipeline on AtoMx™. All results up to the point of export will be in the Seurat object and TileDB array. These are equivalent outputs in different formats. This format is the same for RNA and Protein studies but the format of the raw files folder will be different depending on the analyte. If multiple pipelines from the same study export Raw File there could be TBs of duplicated files. In addition to formats which store data in memory, the AtoMx SIP exports support TileDB formats which access and load data as memory is needed. This format, while less commonly used for single-cell projects, allows for scalability to very high-density analysis. As many CosMx studies will be well in excess of 1 million cells, this new format will enable robust and scalable computations across very large studies without requiring all data to be loaded into memory. By saving data in TileDB arrays it does not need to have all of the data in memory, only the specific parts in use.

The main object in TileDB is a Stack of Matrices, Annotated (SOMA). The TileDB object is a collection of pointers to the RNA counts, normalized RNA counts, negative control probes, and falsecode SOMAs. Having an object of pointers allows for small memory usage. Each SOMA follows the AnnData shape. For protein datasets, count data is currently stored in RNA SOMA.

All matrices in TileDB are stored as sparse matrices. In some embodiments, matrices are counted with targets on rows and cells on columns like a standard Seurat object. In some embodiments, matrices are transposed to look more like AnnData. The raw data cell-by-target expression data are stored in the X slot and can be retrieved and stored in memory. This data is normalized using Pearson Residuals to account for library size factors to ensure that cell specific total transcript abundance and distribution of counts, which may vary some between FOVs and samples. Cell-level metadata are stored in obs slot. It consists of cell information read from the output of CosMx™ SMI.

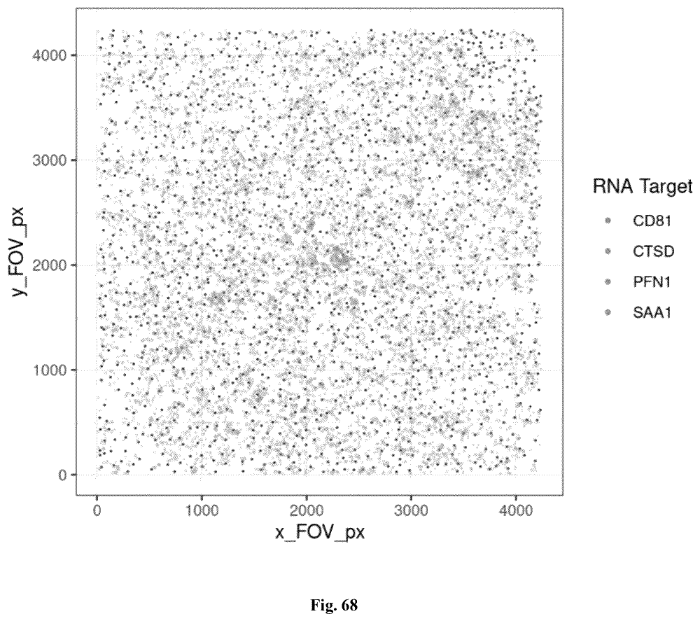

One cell may contain many transcripts, with coordinates of x-y locations of individual transcript. End user can visualize this by plotting transcripts from an individual sample (flow cell 1, normal liver) and a specific region within the tissue (FOV 15). As illustrated in FIG. 68, the centroid of each cell is shown as a black point and each transcript is shown as a colored point. In addition to storing data about count information, the AtoMx SIP enables downstream analysis as well including methods such as differential expression and ligand-receptor analysis. Metadata from these analyses can also be included in the exported results from AtoMx™

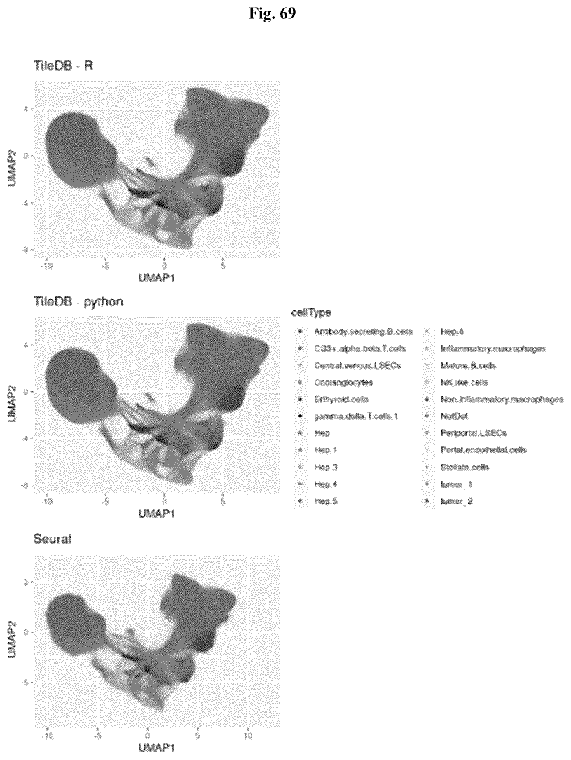

In some embodiments, the TileDB objects can also be read into Seurat objects. In some embodiments, some analysis results of the TileDB objects can be read in individually from the TileDB array. In some embodiments, the TileDB objects can also be read through a Seurat converter that attaches all results to Seurat object. In some embodiments, the TileDB objects can also be read into python. In some embodiments, user can plot a Uniform Manifold Approximation and Projection (UMAP) colored by the cell type metadata. In some embodiments, user can read in UMAP from TileDB python, TileDB R, or Seurat. All of the UMAPs are the same data regardless the route of data read in, as illustrated in FIG. 69.

All analytical modules available on AtoMx™ can provide methods for adding results to a TileDB study. Each study may only use a subset of these modules, and that users can create their own analysis modules which may have different formats and data specifications. All modules can be run with both RNA and protein unless otherwise stated. Data output for any module running before export, will be available in the TileDB and Seurat objects. In some embodiments, the platforms, systems, media, and methods disclosed herein, modules with output data in the TileDB and Seurat objects comprise Spatial Network, Quality Control, Normalization, Principal Component Analysis (PCA), Uniform Manifold Approximation and Projection (UMAP), InSituType (RNA cell typing), CELESTA (Protein cell typing), Nearest Neighbor, Leiden Clustering, Neighborhood Analysis, Spatial Expression Analysis, Cell Type Co-localization, Marker Genes, Ligand-Receptor Analysis (RNA only), Signaling Pathways (RNA only), or Differential Expression (RNA only).

### Collaboration

In some embodiments, the platforms, systems, media, and methods disclosed herein include features and functionality to allows users to collaborate on studies. In various embodiments, subject matter described herein allows users to share within an organization, share with external invited users and trial users, share within a user from different organizations, share data between organizations, and/or conduct federated learning to develop and train AI/ML algorithms. In such embodiments, data sharing and federated AI/ML enables, for example: 1) crowd-sourcing data to fuel NSTG analytics including automated ROI selection, 2) high-throughput AI drug discovery including identifying new gene signatures and/or new targets, and 3) finding individuals with matching morphology and gene profile to identify potential phenotype for a patient.

### EXAMPLES

The following illustrative examples are representative of embodiments of the software applications, systems, and methods described herein and are not meant to be limiting in any way.

### Example 1—Exemplary User Workflow

Open a study (containing 1-N flow cells);
Pipelines → Series of analysis methods
linked together as “modules”;
Create/Edit/Run ;
Visualize Results;
Graphical representations of data (output
of analysis modules);
X-Y, Heatmap, Histogram, Box Plot,
Violin Plot;
Iteration expected (Run a pipeline or
module, visualize results, repeat . . .);
Annotate Data;
Describe data with text or shapes;
Subset data → analyze again;
and
Interact with data for exploratory analysis.

### Example 2—A Spatial Molecular Imager Study of Non-Small Cell Lung Cancer (NSCLC) Formalin Fixed Paraffin Embedded (FFPE) Samples (Spatially Resolved High-Plex RNA Data)

A NanoString Technologies CosMx™ Spatial Molecular Imager was used to profile 960 genes across 5 NSCLC samples, one in triplicate, for 7 total slides and 771,236 cells. The CosMx Spatial Molecular Imager can measure over 1000 genes in a 1 cm² area in each of 2 flow cells, assaying 3 million cells in a single run.

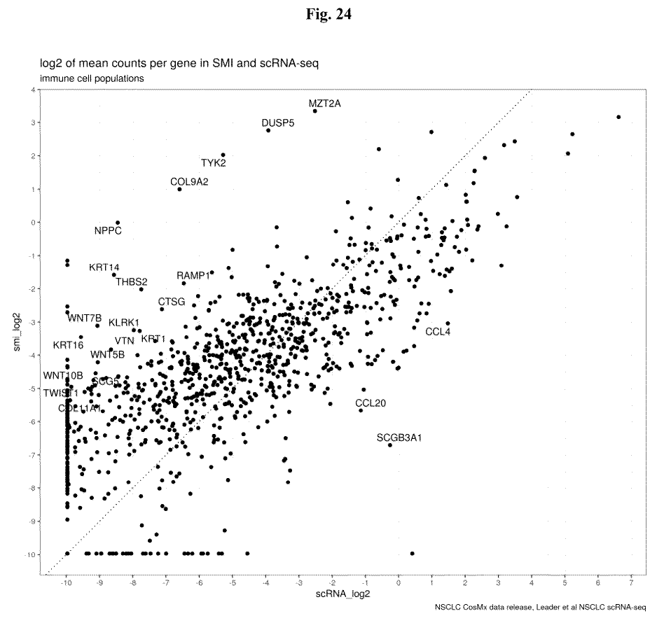

To assess sensitivity, we compared mean signal in CosMx SMI to mean signal in a scRNA-seq dataset from CD45+ cells from NSCLC tumors. Only immune cells were used in the comparison. FIG. 24 shows mean per-cell expression in CosMx SMI vs. scRNA-seq data. Genes below the line had higher average counts in scRNA-seq; genes above the line had higher average counts in CosMx SMI data.

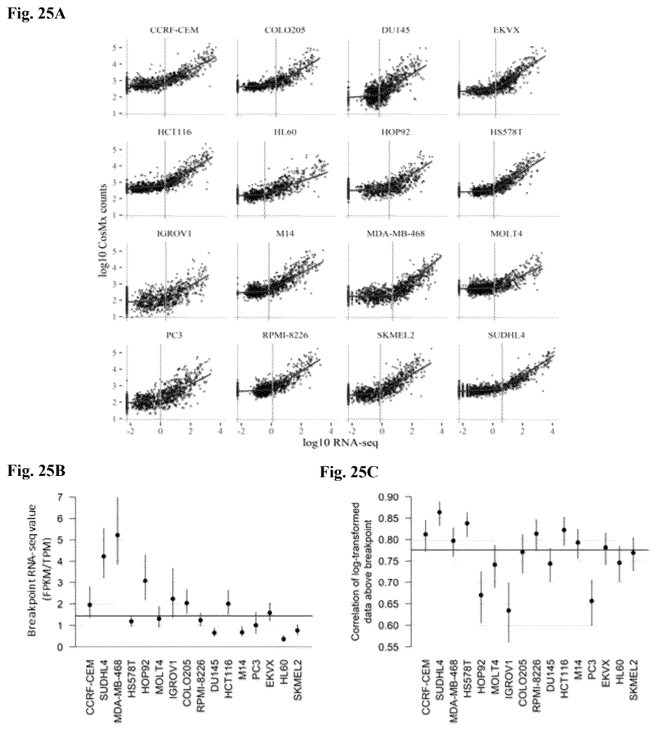

Concordance with RNA-seq was demonstrated in cell lines. 16 Cell lines were profiled with CosMx SMI and bulk RNA-seq. FIG. 25A shows RNA-seq vs. “bulk” CosMx profile. Red lines show breakpoint regression; orange lines mark the breakpoint between the background-dominated data and the signal-dominated data. FIG. 25B shows FPKM values of the breakpoint above which CosMx SMI and RNA-seq are linear. FIG. 25C shows correlation between RNA-seq and CosMx SMI data above the breakpoint.

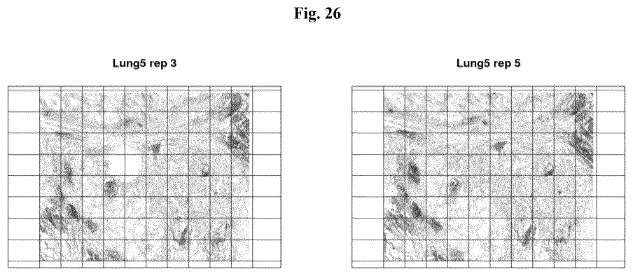

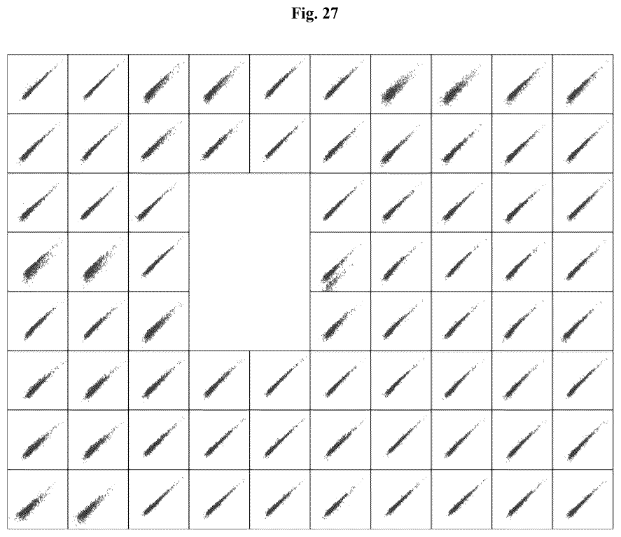

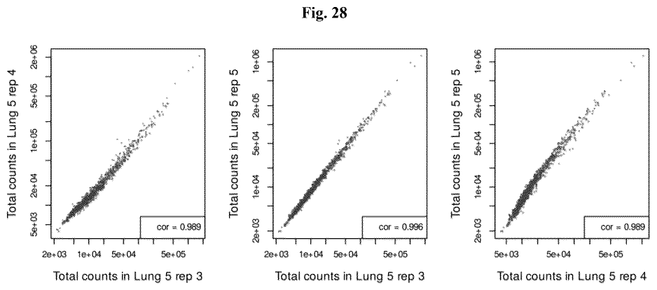

Reproducibility was demonstrated in serial sections. FIG. 26 shows two serial sections of FFPE lung tissue (Lung 5 replicates 3 and 5) was partitioned into a grid. Squares held between 600 and 2,000 cells. FIG. 27 shows concordance between the 980-gene expression profiles of matching grid squares. FIG. 28 shows concordance between “bulk” profiles of 3 replicate sections.

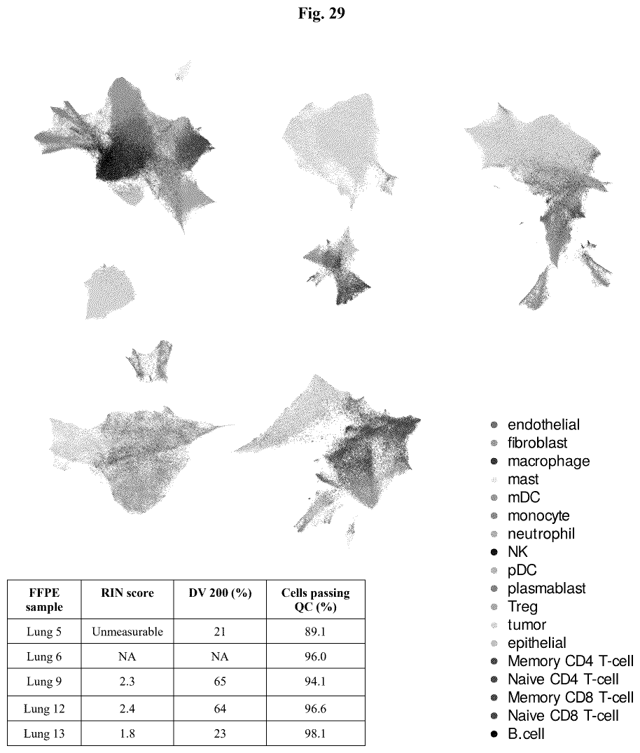

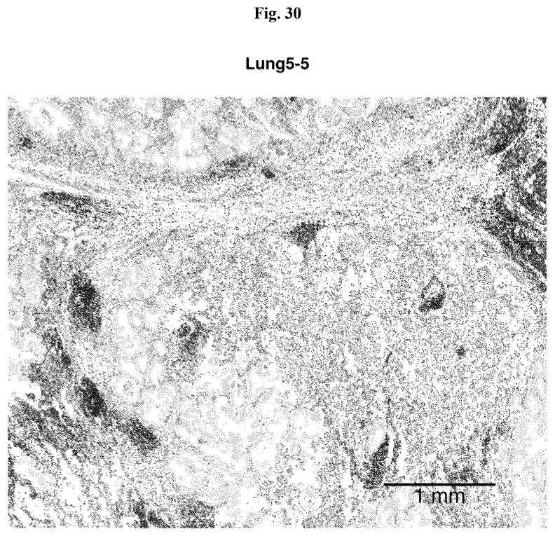

NSCLC cells were imaged in expression space and physical space. See FIGS. 29 and 30.

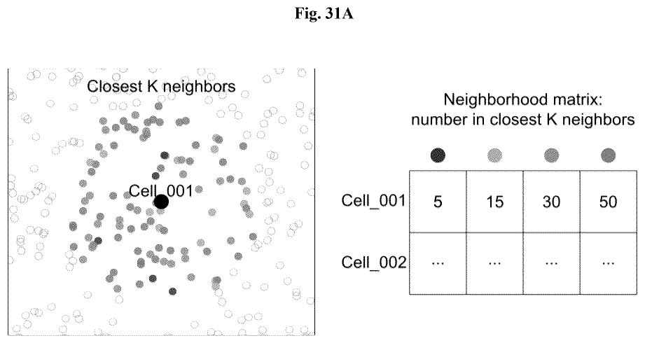

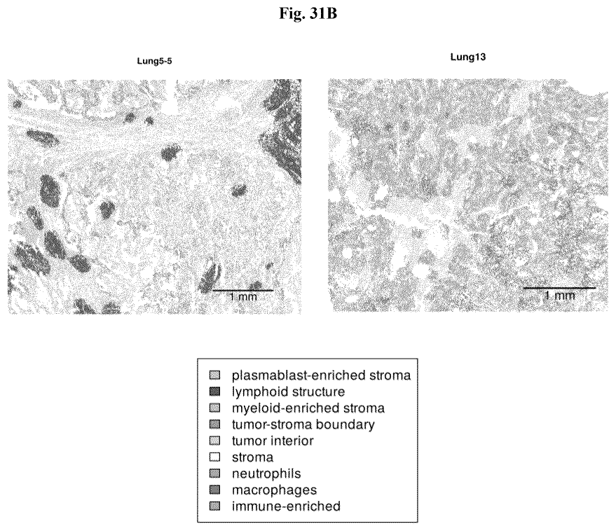

The ecosystems existing within the tissues was investigated. FIGS. 31A and 31B show results pertaining to neighborhood clustering. FIG. 31A shows how each cell's environment was characterized based on the cell types in its neighborhood and cells were clustered based on their neighborhood compositions. FIG. 31B shows neighborhood clustering results in two tissues.

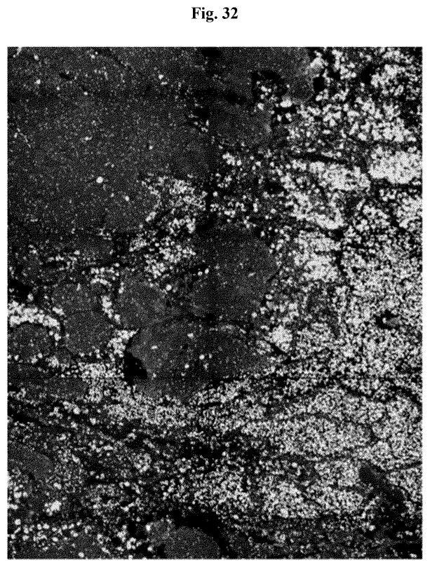

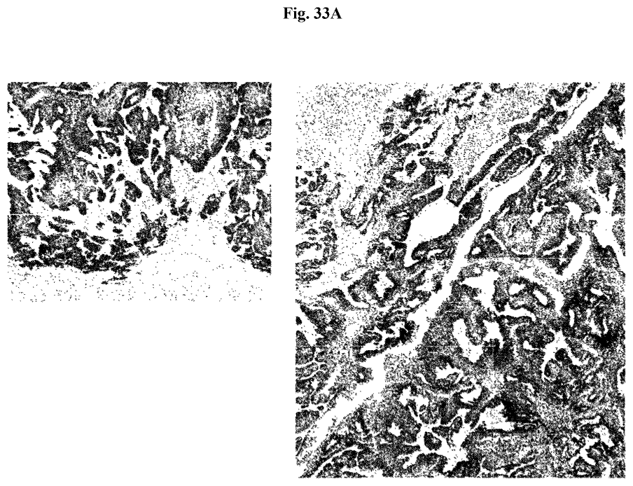

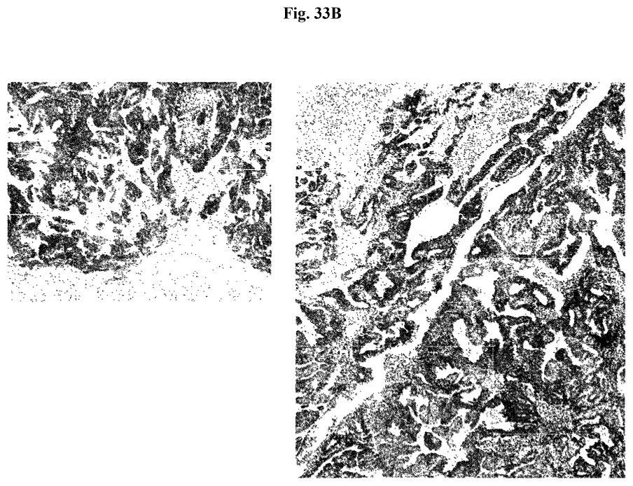

How the cells respond to their environments was investigated. FIG. 32 shows macrophage gene expression changes across the span of tumor “Lung 6.” Yellow dots represent SPP1, a driver of macrophage polarization, up-regulates PD-Li and white dots represent HLA-DQA1, needed for MHC-II antigen-presentation. FIGS. 33A and 33B show spatial dependence of tumor expression. FIG. 33A shows VEGFA expression in tumor cells. FIG. 33B shows HLA-C expression in tumor cells. Expression patterns of VEGFA and HLA-C are both complex and highly spatially ordered.

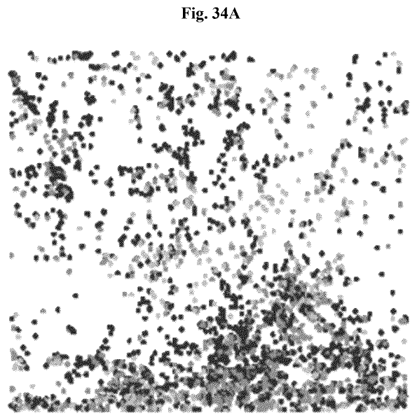

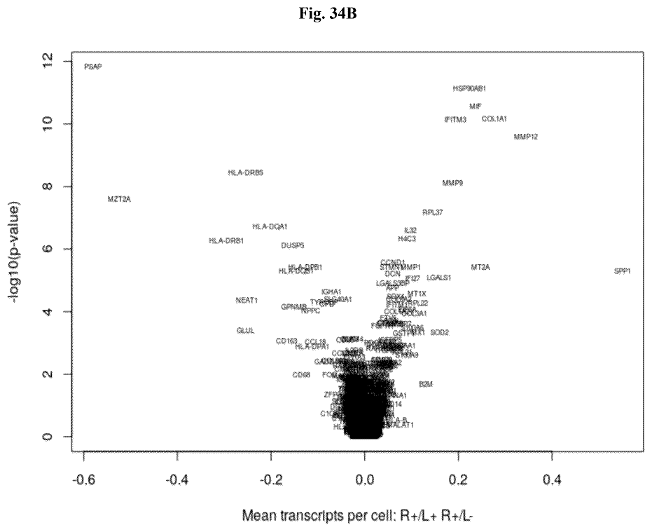

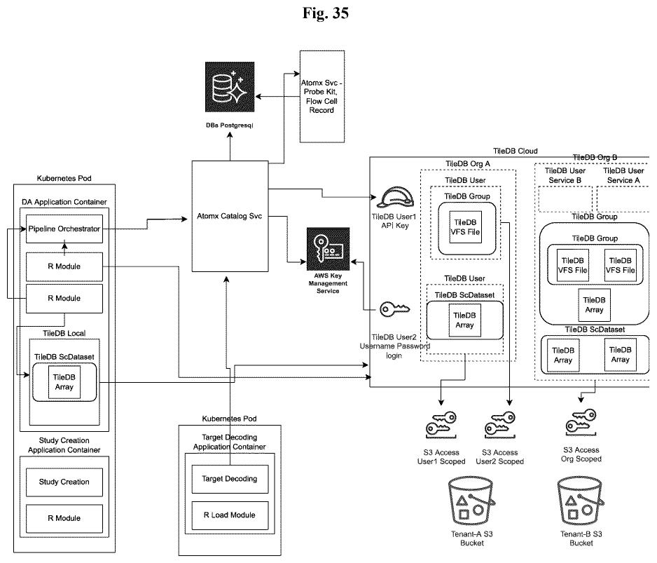

How cells interact with their neighbors was investigated. FIG. 34A shows ligand-receptor signaling analysis. Macrophages were scored for APP→CD74 signaling. Grey represents CD74-macrophages, blue represents CD74+ macrophages with only APP− neighbors, and red represents CD74+ macrophages with APP+ neighbors. FIG. 34B shows cellular response to ligand-receptor signaling. The approach was to perform differential expression comparing CD74+ macrophages with/without APP+ neighbors.

### Example 3—First Exemplary Embodiment

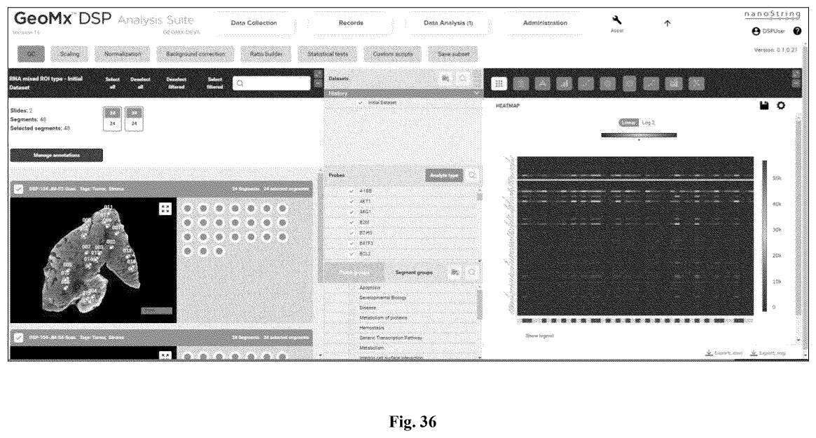

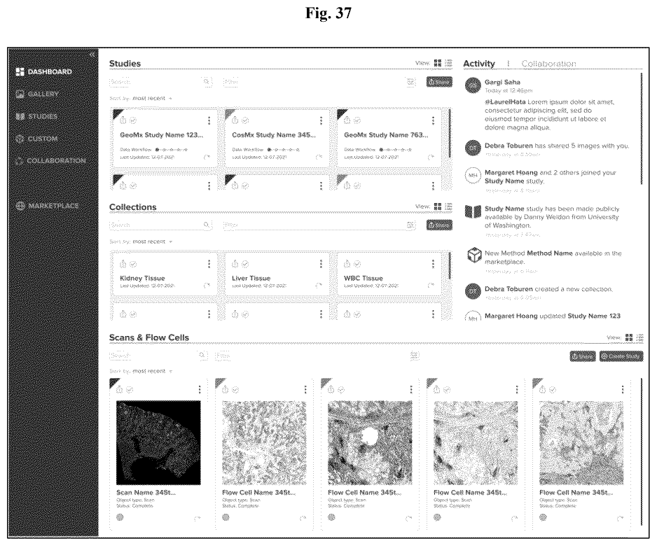

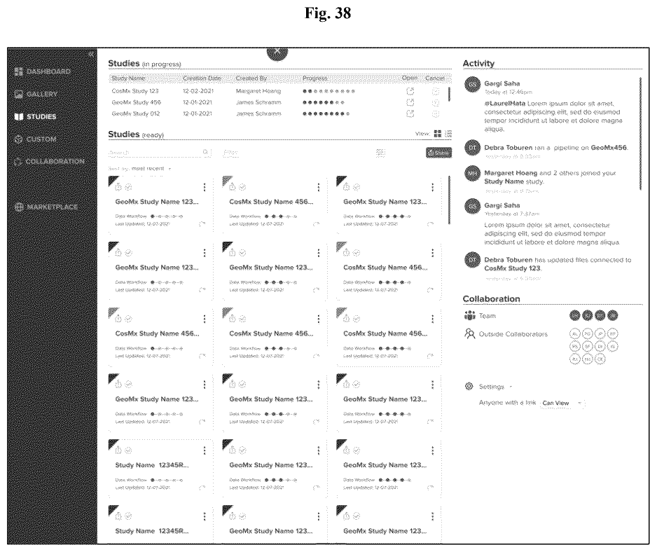

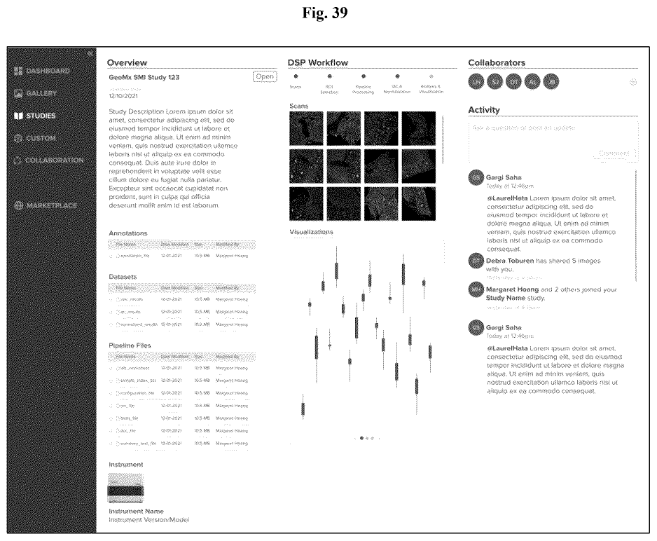

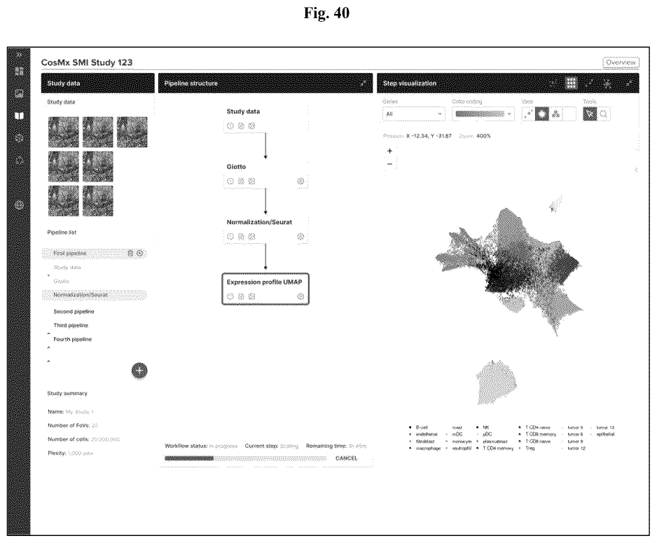

In a first particular non-limiting embodiment, a spatial biology informatics portal described herein includes a data analysis suite that provides on-instrument analysis and visualization. FIG. 36 shows an exemplary data analysis suite including a slide explorer section, a datasets and probes section, as well as multiple analysis tools. In this example, the spatial biology informatics portal further comprises a workspace to manage studies, images and results across laboratory instrument platforms. FIG. 37 shows an exemplary workspace including features and functionality for providing direct access to experimental data, tools for sharing data and results with collaborators around the globe, and streamlined analysis with integrated application specific pipelines. Referring to FIG. 37, the workspace includes navigation providing access to a dashboard, a gallery, a studies screen, a collaboration screen, and a marketplace. Continuing to refer to FIG. 37, a dashboard includes ready access to studies, collections, scans and flow cells associated with a user as well as an activity and collaboration history. Referring to FIG. 38, a studies screen includes a list of studies in progress as well as a list of studies that are ready. For each study, the collaborators are graphically summarized. Referring to FIG. 39, a screen for a particular study includes a consolidated view for the study, including an overview, one or more instruments associated with the study, a workflow, one or more data visualizations, a summary of collaborators, and an activity history. Referring to FIG. 40, a screen for a particular study includes, for the study, a study summary, the study data, a pipeline list providing access to the structure for each pipeline, e.g., a pipeline orchestrator, and an optional visualization of each step in each pipeline. In this example, the pipeline orchestrator allows a user to: 1) create, execute, and save pipelines for future analyses, 2) modify pipelines, 3) add custom modules, and 4) display selected data files, pipelines, and visualizations in an integrated viewer.

### Example 4—Second Exemplary Embodiment

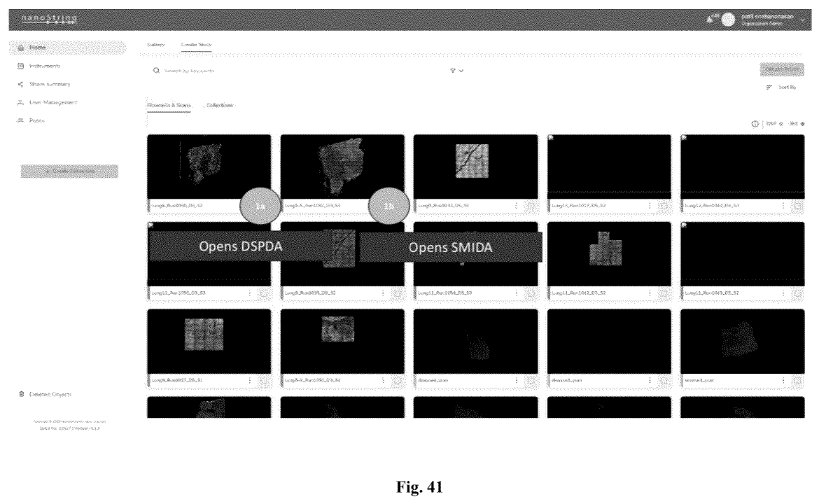

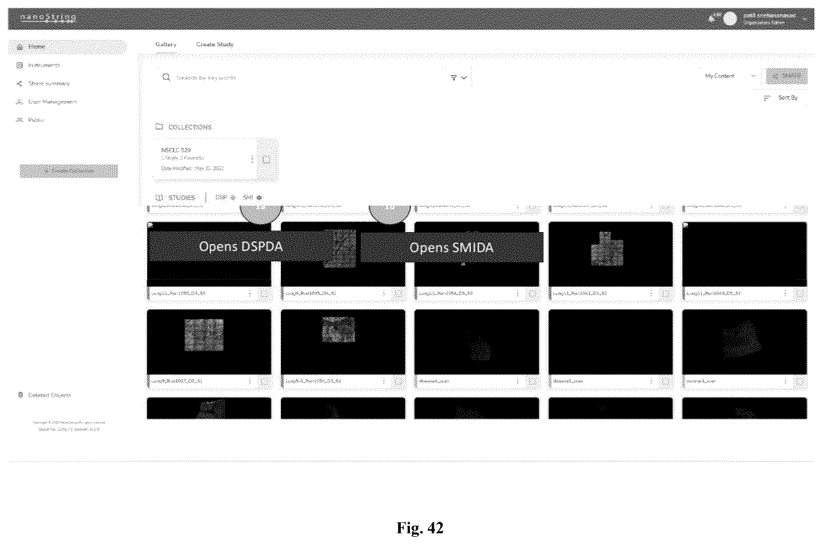

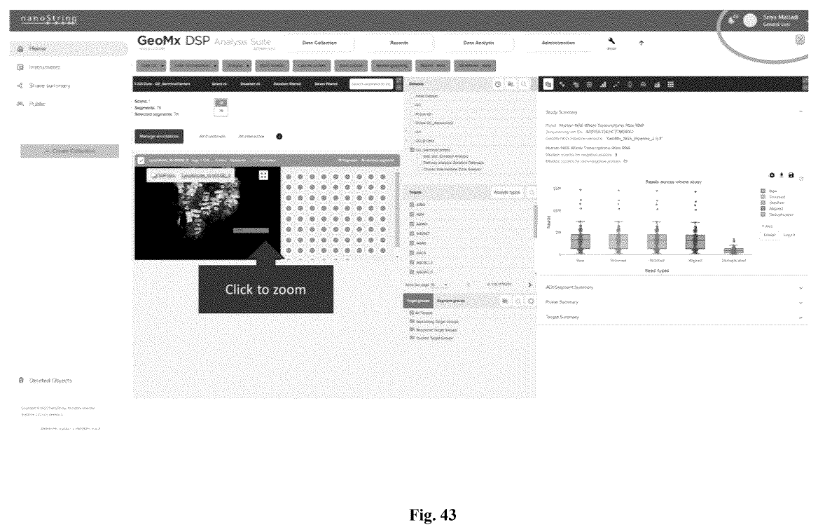

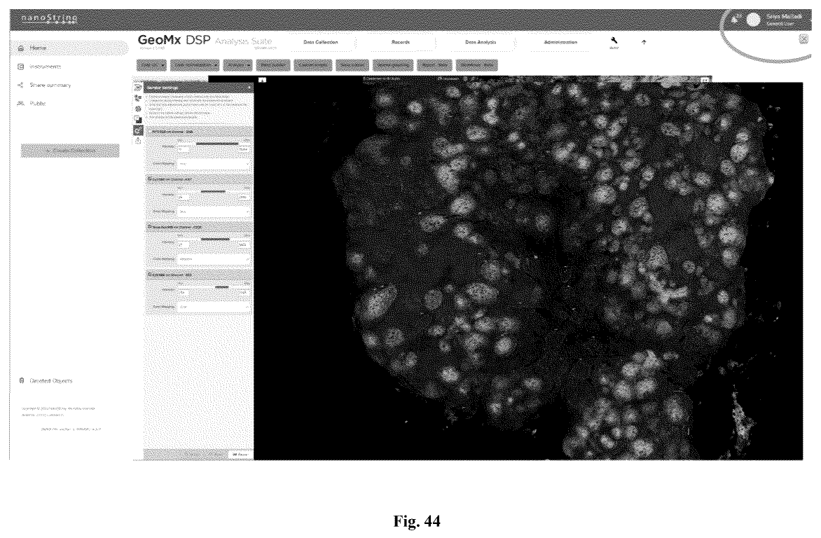

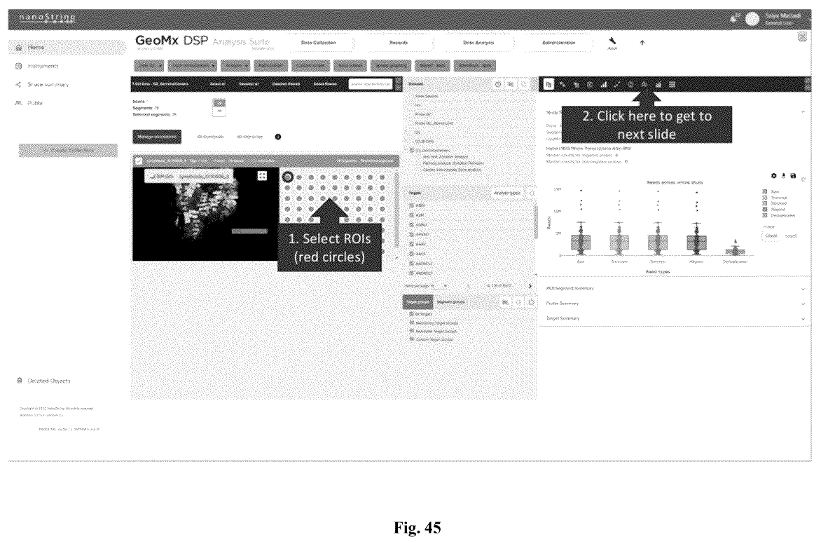

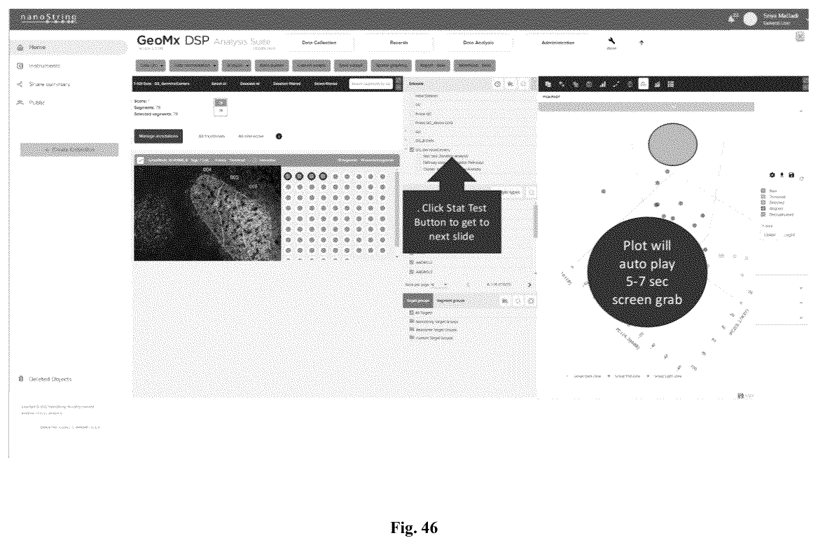

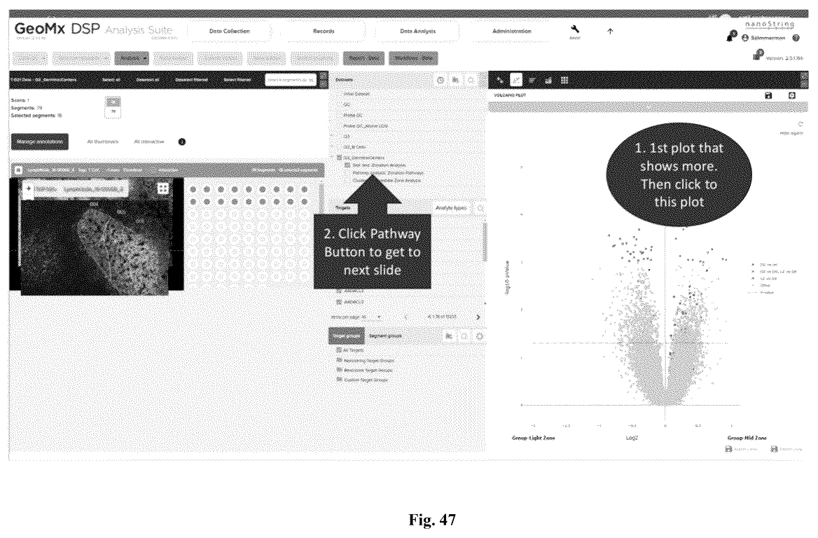

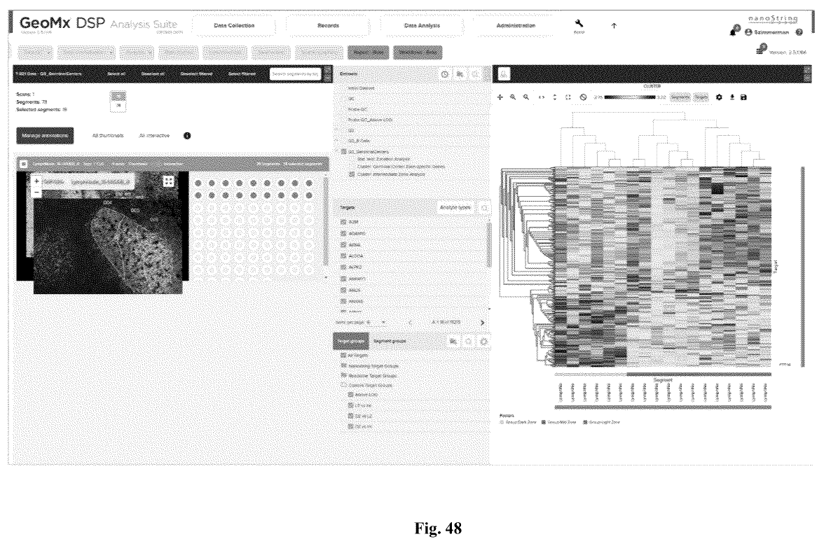

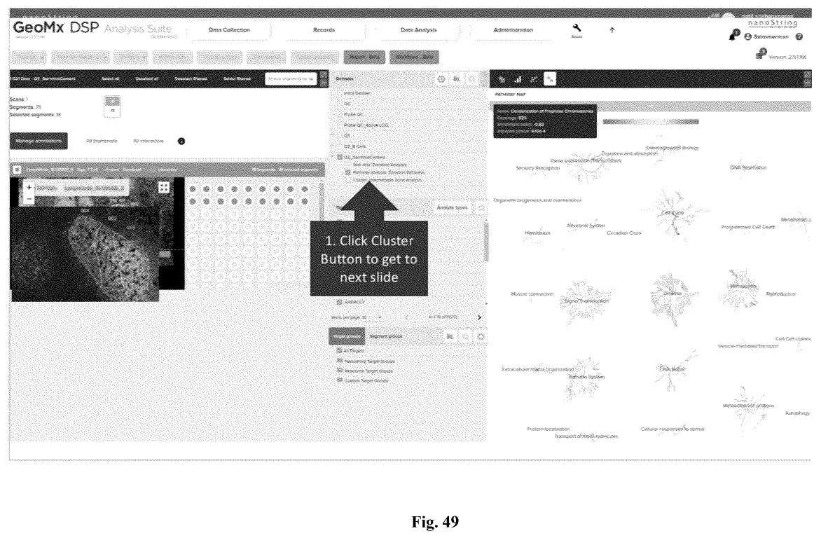

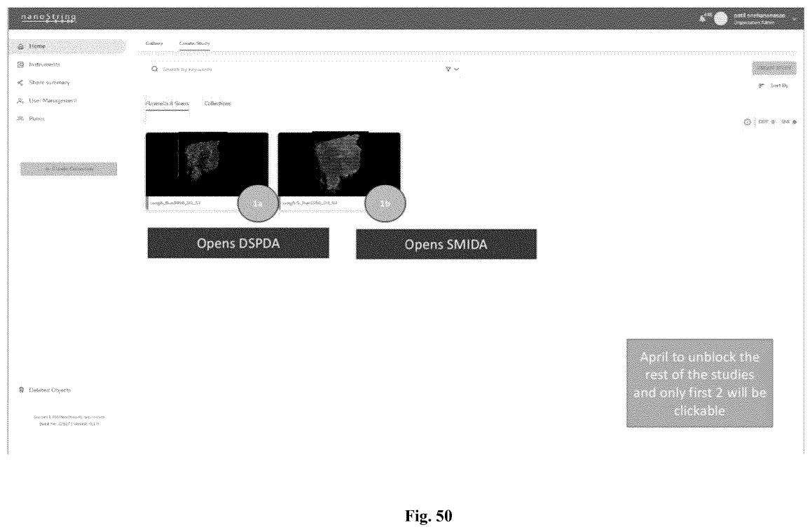

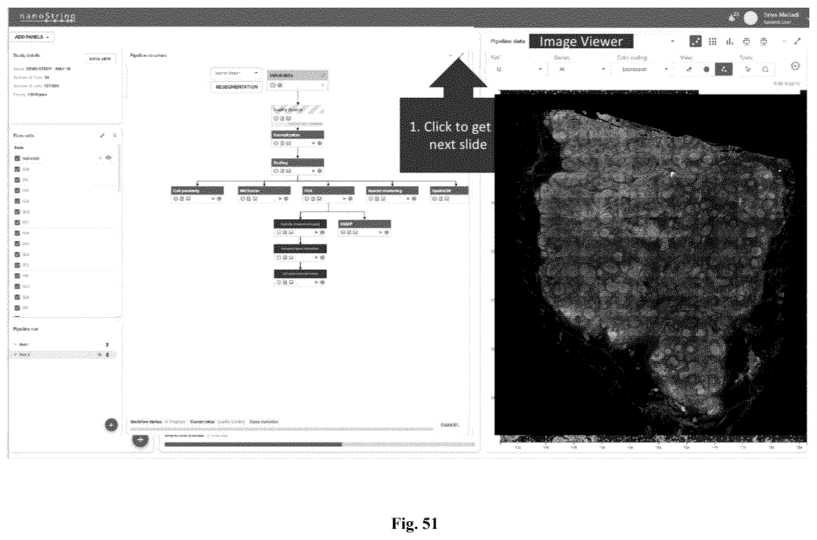

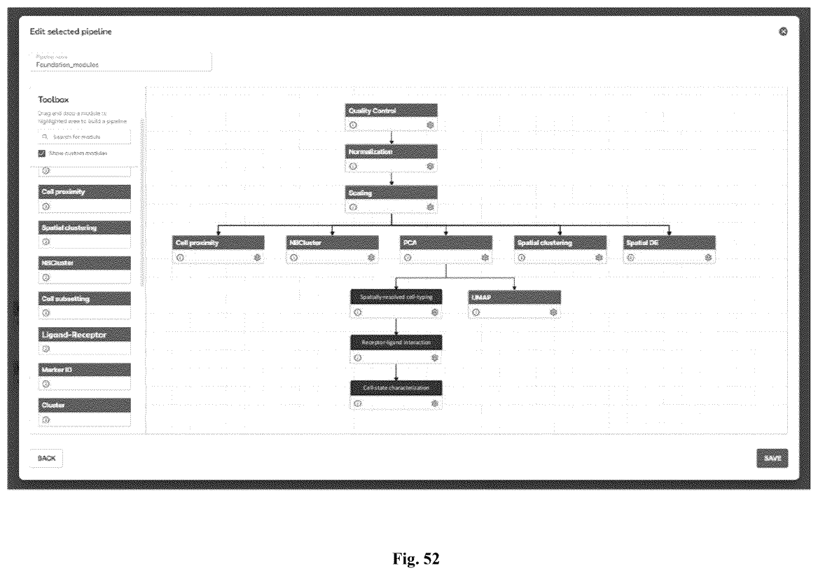

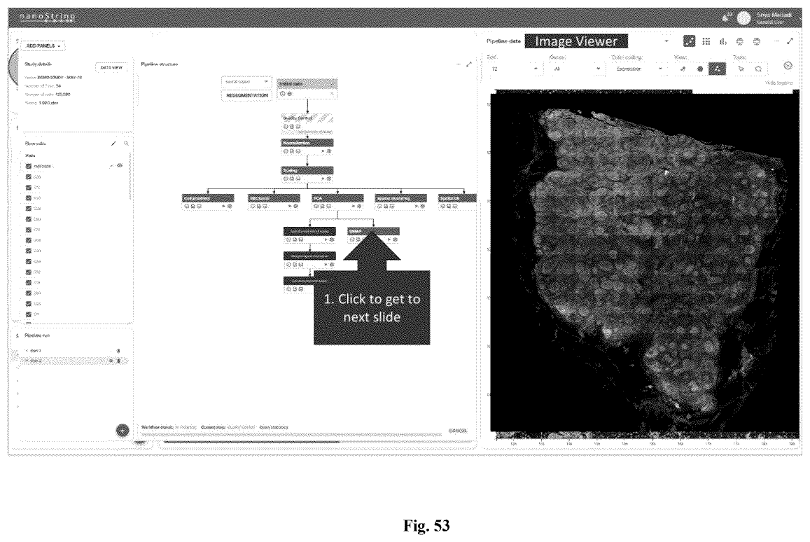

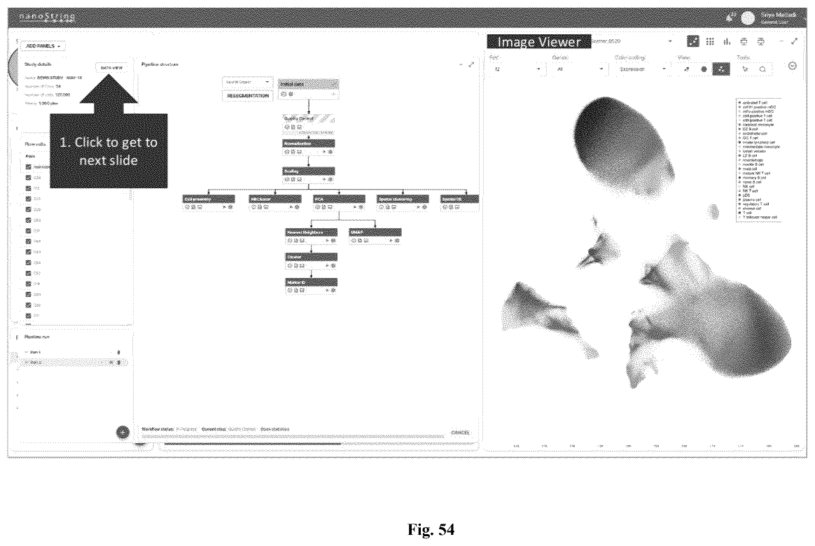

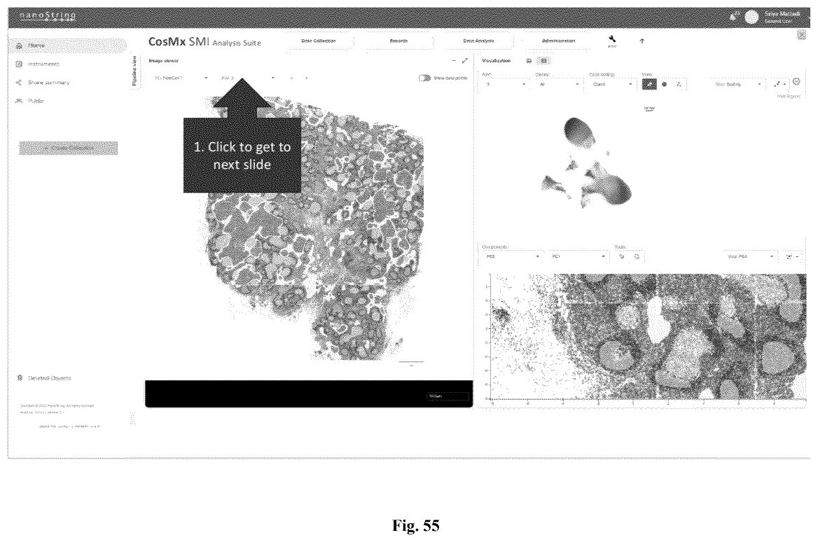

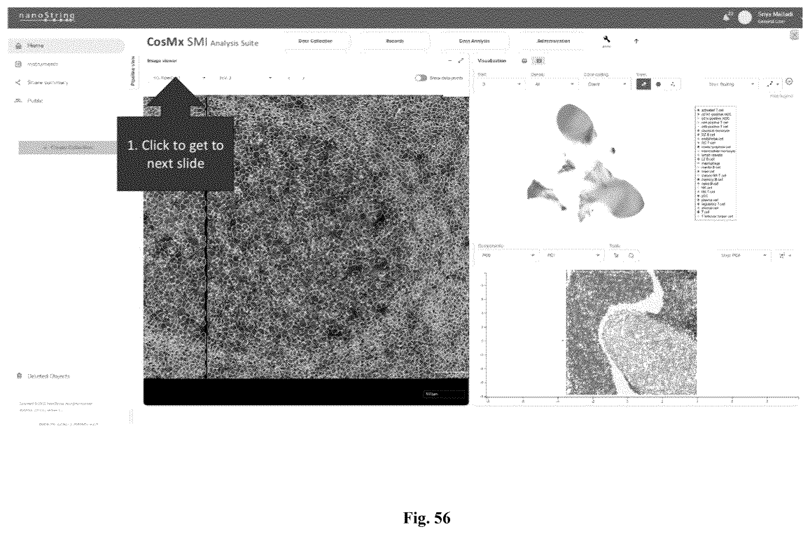

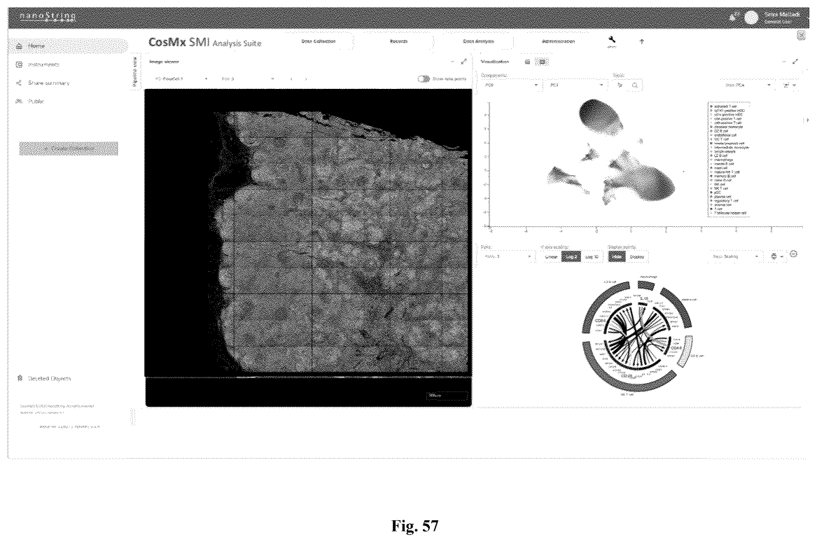

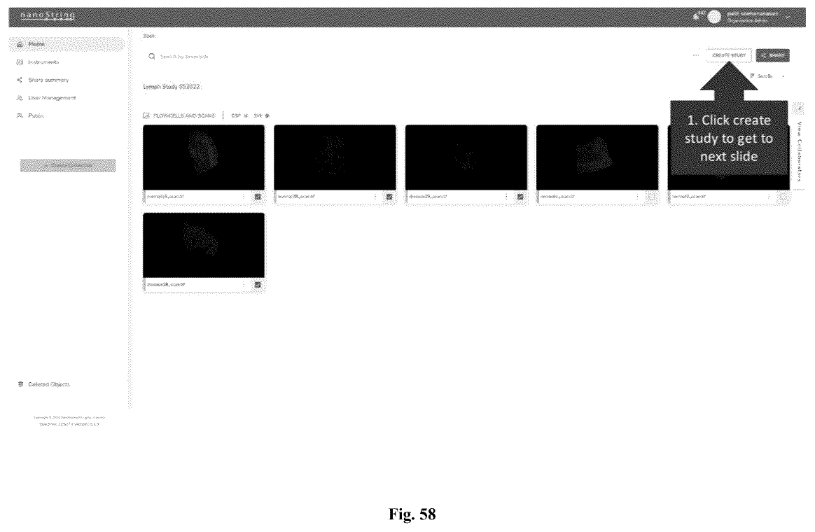

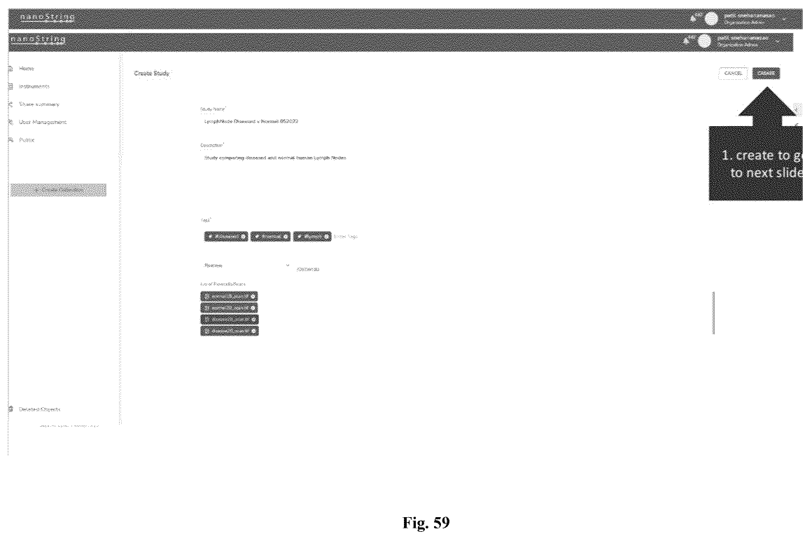

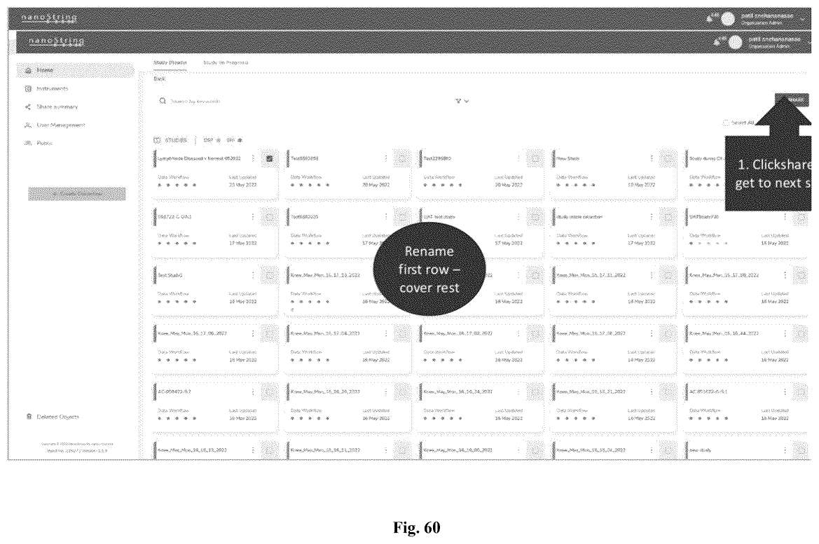

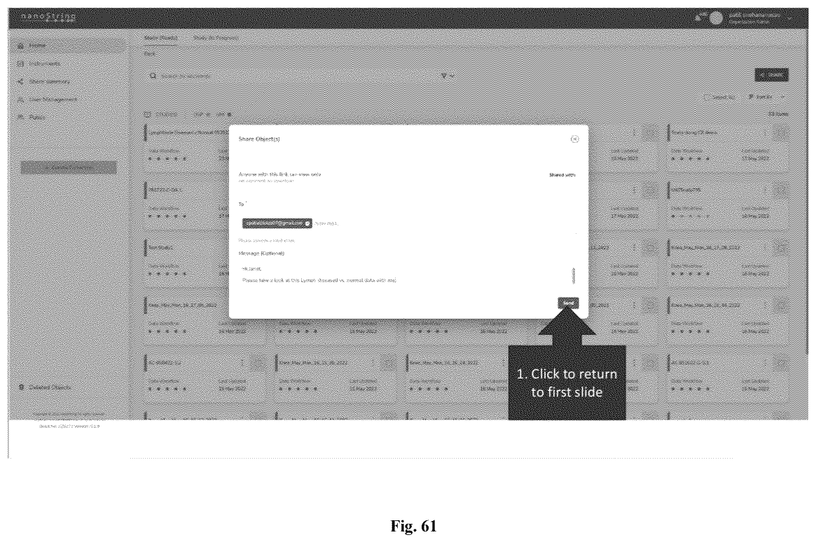

In a second particular non-limiting embodiment, a spatial biology informatics integration portal described herein includes navigation providing access to a home screen, an instruments screen(s), a share summary screen(s), a user management screen(s), and a public screen(s). See FIGS. 41-61.

While preferred embodiments of the present subject matter have been shown and described herein, it will be obvious to those skilled in the art that such embodiments are provided by way of example only. Numerous variations, changes, and substitutions will now occur to those skilled in the art without departing from the present subject matter. It should be understood that various alternatives to the embodiments of the present subject matter described herein may be employed in practicing the present subject matter.

1. A system comprising at least one processor and instructions executable by the at least one processor to provide a spatial biology informatics application comprising:
a) an instrument interface communicatively coupled to one or more laboratory instruments;
b) a software element configured to receive data, directly or indirectly, from the one or more laboratory instruments, wherein the data comprises biological image data and one or more of: genomic, proteomic, metabolomic, metagenomic, phenomic, and transcriptomic data;
c) a software element configured to provide a pipeline orchestrator tool allowing a user to create, edit, manage, and execute analysis pipelines, wherein the pipeline orchestrator tool allows the user to link together modules and run them subsequently or in parallel to transform the received data; and
d) a software element configured to generate a visualization of the received and/or the transformed data.

2. The system of claim 1, wherein the one or more laboratory instruments comprises a DNA sequencer or sequencing platform, a digital spatial profiler, a spatial molecular imager, a RNA expression profiler, or a combination thereof.

3. The system of claim 1, wherein the instrument interface allows the application to perform one or more of: monitoring the one or more laboratory instruments, receiving the data from the one or more laboratory instruments, and sending operating instructions to the one or more laboratory instruments.

4. The system of claim 1, wherein the pipeline orchestrator tool comprises a graphic user interface (GUI) and the user creates and/or edits analysis pipelines by dragging and dropping modules within the GUI.

5. The system of claim 1, wherein the pipeline orchestrator tool allows the user to create, edit, manage, and execute branching analysis pipelines.

6. The system of claim 1, wherein one or more modules available in the pipeline orchestrator tool comprises training a machine learning model and/or applying a machine learning model.

7. (canceled)

8. The system of claim 1, wherein the application further comprises a software element configured to provide a user interface allowing the user to collaborate and share studies.

9. The system of claim 1, wherein the visualization is a three-dimensional (3D) representation.

10. Non-transitory computer-readable storage media encoded with instructions executable by one or more processors to provide a spatial biology informatics application comprising:
a) an instrument interface communicatively coupled to one or more laboratory instruments;
b) a software element configured to receive data, directly or indirectly, from the one or more laboratory instruments, wherein the data comprises biological image data and one or more of: genomic, proteomic, metabolomic, metagenomic, phenomic, and transcriptomic data;
c) a software element configured to provide a pipeline orchestrator tool allowing a user to create, edit, manage, and execute analysis pipelines, wherein the pipeline orchestrator tool allows the user to link together modules and run them subsequently or in parallel to transform the received data; and
d) a software element configured to generate a visualization of the received and/or the transformed data.

11. (canceled)

12. The non-transitory computer-readable storage media of claim 10, wherein the instrument interface allows the application to perform one or more of: monitoring the one or more laboratory instruments, receiving the data from the one or more laboratory instruments, and sending operating instructions to the one or more laboratory instruments.

13. (canceled)

14. The non-transitory computer-readable storage media of claim 10, wherein the pipeline orchestrator tool allows the user to create, edit, manage, and execute branching analysis pipelines.

15. The non-transitory computer-readable storage media of claim 10, wherein one or more modules available in the pipeline orchestrator tool comprises training a machine learning model and/or applying a machine learning model.

16. (canceled)

17. The non-transitory computer-readable storage media of claim 10, wherein the application further comprises a software element configured to provide a user interface allowing the user to collaborate and share studies.

18. (canceled)

19. A computer-implemented method comprising:
a) providing, at a computer, an instrument interface communicatively coupled to one or more laboratory instruments;
b) receiving, at the instrument interface, data from the one or more laboratory instruments, wherein the data comprises biological image data and one or more of: genomic, proteomic, metabolomic, metagenomic, phenomic, and transcriptomic data;
c) providing, at the computer, a pipeline orchestrator tool allowing a user to create, edit, manage, and execute analysis pipelines, wherein the pipeline orchestrator tool allows the user to link together modules and run them subsequently or in parallel to transform the received data; and
d) generating a visualization, by the computer, of the received and/or the transformed data.

20. The method of claim 19, wherein the one or more laboratory instruments comprises a DNA sequencer or sequencing platform, a digital spatial profiler, a spatial molecular imager, a RNA expression profiler, or a combination thereof.

21. The method of claim 19, wherein the instrument interface allows further performance of one or more of: monitoring the one or more laboratory instruments, receiving the data from the one or more laboratory instruments, and sending operating instructions to the one or more laboratory instruments.

22. (canceled)

23. The method of claim 19, wherein the pipeline orchestrator tool allows the user to create, edit, manage, and execute branching analysis pipelines.

24. The method of claim 19, wherein one or more modules available in the pipeline orchestrator tool comprises training a machine learning model and/or applying a machine learning model.

25. (canceled)

26. The method of claim 19, further comprising providing a user interface allowing the user to collaborate and share studies.

27. The method of claim 19, wherein the visualization is a three-dimensional (3D) representation.
